(19) United States
(12) Patent Application Publication
(10) Pub. No.: US 2025/0040845 A1
(43) Pub. Date: Feb. 6, 2025

(54) BACKGROUND INTERFERENCE MITIGATION FOR HIGH SENSITIVITY CREATININE SENSING

(71) Applicant: ABBOTT DIABETES CARE INC., Alameda, CA (US)

(72) Inventors: Cade FOX, Burlingame, CA (US); Tianmei OUYANG, Saratoga, CA (US); Yagya Raj OJHA, Alameda, CA (US); Zenghe LIU, Alameda, CA (US); Jonathan MCCANLESS, Oakland, CA (US); Benjamin FELDMAN, Berkeley, CA (US)

(73) Assignee: ABBOTT DIABETES CARE INC., Alameda, CA (US)

(21) Appl. No.: 18/789,387

(22) Filed: Jul. 30, 2024

Related U.S. Application Data
(60) Provisional application No. 63/516,633, filed on Jul. 31, 2023.

(51) Int. Cl. A61B 5/1486; G01N 27/327

(52) U.S. Cl. CPC A61B 5/14865; G01N 27/3272; G01N 27/3274

(57) ABSTRACT

The present disclosure relates to a creatinine sensor comprising a first working electrode, a creatinine sensing layer on the first working electrode comprising a redox mediator, creatinine amidohydrolase, creatine amidinohydrolase, and sarcosine oxidase, and a hydrophilic polyurethane membrane overcoating the creatinine sensing layer. The creatinine sensor can further comprise a background sensing electrode that does not detect creatinine. The present disclosure further relates to a method for sensing creatinine comprising exposing the creatinine sensor with a background sensing electrode to a fluid; applying a potential to the first and second working electrodes; obtaining a first signal from the first working electrode proportional to a concentration of creatinine and background interference in the fluid; obtaining a second signal from the second working electrode proportional to a concentration of background interference in the fluid; and determining the concentration of creatinine in the fluid by subtracting the second signal from the first signal.

### BACKGROUND

Accurate measurement of creatinine levels in a patient's blood is an important indicator of renal health. Continuous creatinine sensing has the potential to provide valuable medical information for personalized health assessments, including monitoring or scheduling kidney dialysis and monitoring or detecting acute kidney injury. While such measurements are important, in vivo creatinine concentrations tend to be relatively low—often more than 100-fold lower than glucose concentrations. Moreover, electroactive molecules present in biological fluids, such as ascorbic acid, can interact with biosensors to create background interference that can reduce analyte measurement accuracy.

Thus, there is a need for a biosensor with improved creatinine sensitivity and that can reduce background signal interference signal to provide accurate and continuous creatinine monitoring in vivo.

### BRIEF SUMMARY

The present disclosure relates to a creatinine sensor comprising
a first working electrode,
a creatinine sensing layer disposed on at least a portion of the first working electrode, the creatinine sensing layer comprising a redox mediator, creatinine amidohydrolase, creatine amidinohydrolase, and sarcosine oxidase, and
a first hydrophilic polyurethane membrane overcoating at least the creatinine sensing layer.

In some aspects, the first hydrophilic polyurethane membrane is permeable to creatinine.

In some aspects, the first hydrophilic polyurethane membrane is a thermoplastic polyurethane elastomer. In some of these aspects, the first hydrophilic polyurethane has a Shore A hardness of at least about 60A. In some of these aspects, the first hydrophilic polyurethane has a Shore A hardness of at most about 93A.

In some aspects, the first hydrophilic polyurethane is capable of absorbing about 5% to about 25% by weight water. In some aspects, the first hydrophilic polyurethane membrane is not crosslinked.

In some aspects, the sensor further comprises a housing. In some aspects, the sensor further comprises a sensor tail configured for implantation into a tissue, wherein the creatinine sensor is disposed on the sensor tail. In some aspects, the sensor further comprises a reference electrode, a counter electrode, or both a reference electrode and a counter electrode. In some aspects, the sensor further comprises at least one insulation layer. In some aspects, the sensor further comprises a substrate, wherein the creatinine sensor is disposed on the substrate.

In some aspects, the creatinine amidohydrolase, creatine amidinohydrolase, sarcosine oxidase, or any combination thereof is attached to the redox mediator. In some aspects, the redox mediator comprises a polymer and an electron transfer agent. In some aspects, the polymer comprises a backbone comprising poly(4-vinylpyridine), poly(1-vinylimidazole), poly(styrene), poly(thiophene), poly(aniline), poly(pyrrole), poly(acetylene), or any combination thereof. In some aspects, the polymer comprises a polymer or copolymer repeat unit comprising at least one pendant pyridinyl group, at least one pendant imidazolyl group, or both at least one pendant pyridinyl and at least one pendant imidazolyl group. In some aspects, the electron transfer agent comprises a transition metal complex. In some of these aspects, the transition metal complex comprises osmium, ruthenium, iron, cobalt, or any combination thereof. In some of these aspects, the transition metal complex is an osmium transition metal complex comprising one or more ligands, wherein at least one ligand comprises a nitrogen-containing heterocycle.

In some aspects, the redox mediator comprises an osmium complex bonded to a poly(vinylpyridine)-based polymer. In some of these aspects, the polymer is crosslinked with a crosslinking agent. In some aspects, the crosslinking agent is a polyepoxide, cyanuric chloride, N-hydroxysuccinimide, an imidoester, epichlorohydrin, or any combination thereof. In some aspects, the crosslinking agent is a polyethylene glycol diglycidylether (PEGDGE).

In some aspects, the creatinine sensing layer is continuous. In some aspects, the creatinine sensing layer is discontinuous.

In some aspects, the creatinine sensor further comprises a background sensing electrode comprising
a second working electrode,
a background sensing layer that does not detect creatinine disposed on at least a portion of the second working electrode, the background sensing layer comprising the redox mediator, optionally creatine amidinohydrolase, and optionally sarcosine oxidase, and
a second hydrophilic polyurethane membrane overcoating at least the background sensing layer,
wherein the background sensing layer does not comprise creatinine amidohydrolase.

In some aspects, the second hydrophilic polyurethane membrane is a thermoplastic polyurethane elastomer. In some aspects, the second hydrophilic polyurethane has a Shore A hardness of at least about 60A. In some aspects, the second hydrophilic polyurethane has a Shore A hardness of at most about 93A.

In some aspects, the second hydrophilic polyurethane is capable of absorbing about 5% to about 25% by weight water. In some aspects, the second hydrophilic polyurethane membrane is not crosslinked.

The present disclosure further relates to a method for sensing creatinine.

In some aspects, the method comprises exposing the creatinine sensor to a fluid comprising creatinine; applying a potential to the first working electrode with a creatinine sensing layer; obtaining a first signal that is proportional to a concentration of creatinine and background interference in the fluid; and correlating the first signal to the concentration of creatinine in the fluid.

In some aspects in which a second, background sensing electrode is present in the creatinine sensor, the method for sensing creatinine comprises exposing the creatinine sensor to a fluid comprising creatinine; applying a potential to the first working electrode with a creatinine sensing layer and second working electrode with a background sensing layer; obtaining a first signal from the first working electrode that is proportional to a concentration of creatinine and background interference in the fluid; obtaining a second signal from the second working electrode that is proportional to a concentration of background interference in the fluid; and determining the concentration of creatinine in the fluid by subtracting the second signal from the first signal.

In some aspects of these methods, the potential applied is less than +40 mV vs Ag/AgCl. In some aspects, the potential applied is about +5 mV to about −125 mV vs Ag/AgCl. In some aspects, the potential applied is about −80 mV vs Ag/AgCl.

Additional aspects and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS/FIGURES

FIG. 1 shows a diagram of an illustrative sensing system that can incorporate an analyte sensor of the present disclosure.

FIGS. 2A-2C show cross-sectional diagrams of analyte sensors including a single sensing layer.

FIGS. 3A-3C show cross-sectional diagrams of analyte sensors including two sensing layers.

FIG. 4 shows a cross-sectional diagram of an analyte sensor including two sensing layers.

FIGS. 5A-5C show perspective views of analyte sensors including two sensing layers upon separate working electrodes.

FIG. 6 shows a scheme of how certain enzymes can interact with creatinine and the working electrode.

FIG. 7 shows a graph of current (pA) versus time (days) for two types (2-pass and 4-pass) of blank sensors (n=2): 2-pass Blank 1, 2-pass Blank 2, 4-pass Blank 1, and 4-pass Blank 2.

FIGS. 8A and 8B show a sensor current (nA) versus time (hours) plot of an exemplary creatinine sensor of the present disclosure comprising a hydrophilic aliphatic polyurethane membrane (FIG. 8A) compared to a creatinine sensor comprising a crosslinked poly(4-vinylpyridine) (PVP) membrane or PVP+NAFION™ membrane (FIG. 8B).

FIG. 9 shows a sensor current (nA) versus time (hours) plot of a blank (background) sensor with different sensing potentials applied: +40 mV, −20 mV, −50 mV, and −80 mV, each relative to a Ag/AgCl reference.

FIG. 10 shows creatinine response as a function of slope (nA/mM) versus sensing potential (mV) of a blank sensor applied with +40 mV, −20 mV, −50 mV, and −80 mV, each relative to a Ag/AgCl reference.

FIG. 11 shows a sensor current (nA) versus time (hours) plot of a dual channel sensor: creatinine sensing and blank (background) sensing applied to a human serum sample at −80 mV vs. Ag/AgCl and aliquots of 0.1 mM creatinine added at various time points.

FIG. 12 shows a sensor current (nA) versus time (days) plot of a dual channel sensor: creatinine sensing and blank (background) sensing applied to a human serum sample at −80 mV vs. Ag/AgCl.

### DETAILED DESCRIPTION

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a.” “an,” and “the” are used herein to refer to one or to more than one (i.e., to at least one) of the grammatical object of the article. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 10% (e.g., up to 5% or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given aspect disclosed herein “comprises” certain elements, it should be understood that present disclosure also specifically contemplates and discloses aspects that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an aspect are included.

As used herein, the terms “consists of,” “consisting of,” and the like are to be construed as closed terms, such that an aspect “consisting of” a particular set of elements excludes any element, step, or ingredient not specified in the aspect.

As used herein, an “analyte” is an enzyme substrate that is subject to be measured or detected. The analyte can be from, for example, a biofluid and can be tested in vivo, ex vivo, or in vitro. In typical aspects herein, the analyte is creatinine.

As used herein, the term “background interferents” or “interferents” are substances in an assayed sample that can prevent a desired analyte (e.g., creatinine) from being accurately measured. Common interferents include, e.g., but are not limited to ascorbic acid, uric acid, homovanillic acid, 5-hydroxy-tryptamine, catecholamines (e.g., dopamine, noradrenaline and their major metabolites, such as 3,4-dihydroxyphenylacetic acid, 3-methoxytyramine), indolamines, drug metabolites, fibrinogen, proteins, cells (e.g., white blood cells, red blood cells), metal ions (e.g., copper ions, mercury ions), and combinations thereof.

As used herein, a “biofluid” is any bodily fluid or bodily fluid derivative in which the analyte can be measured. Examples of biofluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears. In certain aspects, the biological fluid is dermal fluid or interstitial fluid.

As used herein, a“counter electrode” refers to an electrode paired with the working electrode, through which passes a current equal in magnitude and opposite in sign to the current passing through the working electrode. In the context of aspects of the present disclosure, the term “counter electrode” includes both a) true counter electrodes and b) counter electrodes that also function as reference electrodes (i.e., counter/reference electrodes), unless otherwise indicated.

As used herein, “crosslinking agent” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinking at the same time.

As used herein, “electrolysis” is the electrooxidation or electroreduction of a compound either directly at an electrode or via one or more electron transfer agents.

As used herein, an “electron transfer agent” is a compound that carries electrons between the analyte and the working electrode, either directly, or in cooperation with other electron transfer agents. One example of an electron transfer agent is a redox mediator.

As used herein, components are “immobilized” or “attached” to a polymer and/or a sensor, for example, when the components are entrapped on, entrapped within, covalently bound, ionically bound, electrostatically bound, or coordinatively bound to constituents of a polymer, a sol-gel matric, membrane, and/or sensor, which reduces or precludes mobility.

As used herein, the term “membrane solution” is a solution that contains the components for forming the membrane, including. e.g., polymer (e.g., hydrophilic polyurethane) and a solvent.

As used herein, a “non-leachable” compound, or a compound that is “non-leachably disposed” is meant to define a compound that is affixed on the sensor such that it does not substantially diffuse away from the sensing layer of the working electrode for the period in which the sensor is used (e.g., the period in which the sensor is implanted in a patient or measuring a sample).

As used herein, the term “patient” refers to a living animal, and thus encompasses a living mammal and a living human, for example. The term “user” can be used herein as a term that encompasses the term “patient.”

As used herein, the term “precursor polymer” refers to the starting polymer before the various modifier groups are attached to form a modified polymer.

A “reactive group” is a functional group of a molecule (e.g., a polymer, a crosslinking agent, an enzyme) that is capable of reacting with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

As used herein, a “reference electrode” includes both a) true reference electrodes and b) reference electrodes that also function as counter electrodes (i.e., counter/reference electrodes), unless otherwise indicated.

As used herein, a “sensing layer” is a component of the sensor including constituents that facilitate the electrolysis of the analyte. The sensing layer can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some aspects of the present disclosure, a sensor includes a sensing layer that is non-leachably disposed in proximity to or on the working electrode. A sensing layer can have more than one sensing element making up the analyte detection area disposed on the working electrode.

As used herein, a “sensing element” is an application or region of an analyte-specific enzyme disposed with the sensing layer. As such, a sensing element is capable of interacting with the analyte. In some aspects, the sensing element includes an analyte-specific enzyme and an electron transfer agent (e.g., electron transfer agent). In some aspects, the sensing element includes an analyte specific enzyme, a redox mediator, and a crosslinking agent.

As used herein, a “sensor” is a device configured to detect the presence and/or measure the level of an analyte in a sample via electrochemical oxidation and reduction reactions on the sensor. These reactions are transduced to an electrical signal that can be correlated to an amount, concentration, or level of an analyte in the sample.

A “substituted” functional group (e.g., substituted alkyl, alkenyl, alkoxy, aryl) includes at least one substituent (e.g., 1, 2, 3, 4, or 5) that can be, for example, halo, alkoxy, mercapto, aryl, alkoxycarbonyl, alkylaminocarbonyl, dialkylaminocarbonyl, hydroxy, amino, alkylamino, dialkylamino, trialkylammonium, alkanoylanino, arylcarboxamido, hydrazino, alkylthio, alkenyl, and reactive groups.

As used herein, the term “working electrode” is an electrode at which the analyte (or a second compound whose level depends on the level of the analyte) is electrooxidized or electroreduced with or without the agency of an electron transfer agent.

As used herein, the term “C₆₋₃₀ aryl” refers to an aromatic compound comprising a mono-, bi-, or tricyclic carbocyclic ring system having one, two, or three aromatic rings, for example, phenyl, naphthyl, anthracenyl, or biphenyl. The aromatic compound generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2π electrons, according to Hückel's Rule, wherein n=1, 2, or 3.

As used herein, the term “halo” refers to a radical of a halogen, i.e., F, Cl, Br, or I.

As used herein, the term “C₁₋₆ alkyl” refers to a straight-chain or branched alkyl substituent containing from, for example, from about 1 to about 6 carbon atoms, e.g., from about 1 to about 4 carbon atoms or about 1 to about 3 carbons. Examples of alkyl group include, but are not limited to, methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as e.g., C₁₋₆ haloalkyl (e.g., -trifluoromethyl (—CF₃)).

As used herein, the term “C₂₋₆ alkenyl” refers to a linear alkenyl substituent containing from, for example, 2 to about 6 carbon atoms (branched alkenyls are about 3 to about 6 carbons atoms). In accordance with an aspect, the alkenyl group is a C₂₋₄ alkenyl. Examples of alkenyl group include, but are not limited to, ethenyl, allyl, 2-propenyl, I-butenyl, 2-butenyl, 1-pentenyl, 2-pentenyl, 3-pentenyl, 1-hexenyl, and the like.

As used herein, the term “C₂₋₆ alkynyl” refers to a linear alkynyl substituent containing from, for example, 2 to about 6 carbon atoms (branched alkynyls are about 3 to about 6 carbons atoms). In accordance with an aspect, the alkynyl group is a C₂₋₄ alkynyl. Examples of alkynyl group include, but are not limited to, ethynyl, propynyl, 1-butynyl, 2-butynyl, 1-pentynyl, 2-pentynyl, 3-pentynyl, 1-hexynyl, and the like.

As used herein, the term “hydroxy” refers to —OH.

As used herein, the term “nitro” refers to —NO₂.

As used herein, the term “cyano” refers to —CN.

As used herein, the term “amino” refers to —NH₂. The terms mono- and di-C₁₋₆ alkylamino refer to a nitrogen bonded to one or two C₁₋₆ alkyl groups, respectively, i.e., —NHR or —NRR′, in which R and R′ are the same or different C₁₋₆ alkyl groups.

As used herein, the term “C₁₋₆ alkoxy” refers to a C₁₋₆ alkyl group bonded to an oxygen, i.e., —OR, in which R is a C₁₋₆ alkyl group.

As used herein, the term “C₆₋₁₀ aryloxy” refers to an aryl group bonded to an oxygen, i.e., —O(Ar), in which Ar is a C₆₋₁₀ aryl group.

As used herein, the term “aralkoxy” refers to the group —OR(Ar), in which R is an C₁₋₆ alkyl group and Ar is a C₆₋₁₀ aryl group.

As used herein, the term “carboxy” refers to —C(O)OH.

As used herein, the term “C₁₋₆ alkylcarboxy” refers to a carboxy group wherein the hydrogen bound to the carboxy group has been replaced with a C₁₋₆ alkyl group, i.e., —C(O)OR, wherein R is an C₁₋₆ alkyl group.

As used herein, the term “amido” refers to the structure —C(O)NH or —NHC(O). The term “C₁₋₆ alkylamido” refers to —C(O)NR or —NRC(O), wherein R is C₁₋₆ alkyl.

As used herein, the term “C₁₋₆ haloalkylamido” refers to a C₁₋₆ alkylamido group in which the C₁₋₆ alkyl group is substituted with 1, 2, or 3 halo groups, as described herein.

As used herein, the term “heteroaryl” refers to an aromatic compound, as described herein, containing a 5 or 6 membered ring in which 1 or 2 carbons have been replaced with nitrogen, sulfur, and/or oxygen. Examples of heteroaryl include, but are not limited to, pyridinyl, furanyl, pyrrolyl, quinolinyl, thiophenyl, indolyl, oxazolyl, isoxazolyl, pyrazolyl, imidazolyl, thiazolyl, isothiazolyl, 1,3,4-thiadiazolyl, pyridazinyl, pyrimidinyl, pyrazinyl, and triazinyl.

As used herein, the term “heterocycloalkyl” refers to a monocyclic, bicyclic, or spiro ring system containing 3 to 7 carbon atom ring members and 1, 2, or 3 other atoms selected from nitrogen, sulfur, and/or oxygen. Examples of such heterocycloalkyl rings include, but are not limited to, aziridinyl, oxiranyl, thiazolinyl, imidazolidinyl, piperazinyl, homopiperazinyl, pyrrolinyl, pyrrolidinyl, tetrahydrofuranyl, tetrahydrothiofuranyl, pyranyl, tetrahydropyranyl, piperidinyl, and morpholinyl.

### Sensors, Compositions, and Methods of the Disclosure

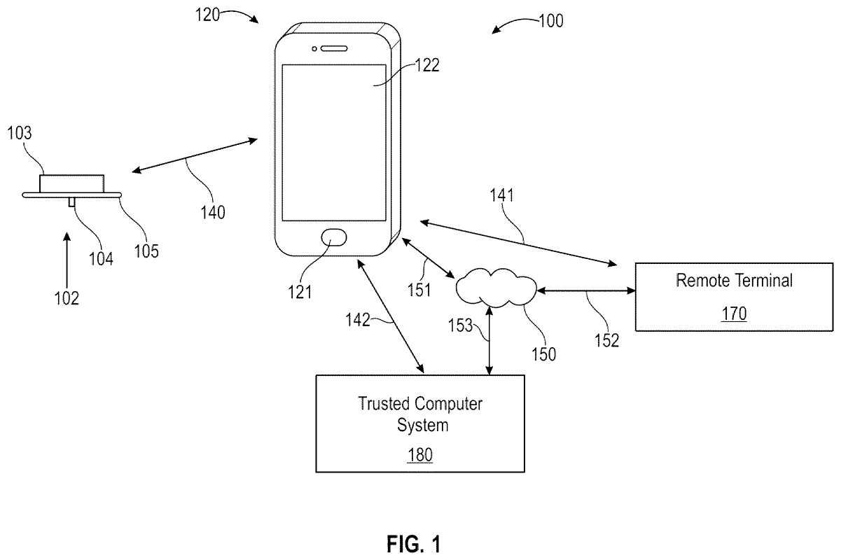

Before describing the analyte sensors of the present disclosure and their components in further detail, a brief overview of suitable in vivo analyte sensor configurations and sensor systems employing the analyte sensors will be provided so that the aspects of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to certain aspects. Reader device 120 can be a multi-purpose smartphone or a dedicated electronic reader instrument. While only one reader device 120 is shown, multiple reader devices 120 can be present in certain instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain aspects, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain aspects, by individuals other than a primary user who have an interest in the user's analyte levels. Reader device 120 can include display 122 and optional input component 121. Display 122 can include a touch-screen interface, according to certain aspects.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain aspects.

Sensor 104 is adapted to be at least partially inserted into a tissue of interest, such as within the dermal or subcutaneous layer of the skin. Sensor 104 can include a sensor tail of sufficient length for insertion to a desired depth in a given tissue. The sensor tail can include at least one working electrode. In certain configurations, the sensor tail can include a sensing layer for detecting an analyte (e.g., creatinine). A counter electrode can be present in combination with the at least one working electrode. Particular electrode configurations upon the sensor tail are described in more detail below.

The sensing layer can be configured for detecting a particular analyte (e.g., creatinine). For example, but not by way of limitation, the disclosed analyte sensors include at least one sensing layer configured to detect an analyte (e.g., creatinine).

In certain aspects of the present disclosure, an analytes (e.g., creatinine) can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain particular aspects, analyte sensors of the present disclosure can be adapted for assaying dermal fluid or interstitial fluid to determine a concentration of one or more analytes in vivo. In certain aspects, the biological fluid is interstitial fluid.

Referring still to FIG. 1, sensor 104 can automatically forward data to reader device 120. For example but not by the way of limitation, analyte concentration data (i.e., glucose concentration) can be communicated automatically and periodically, such as at a certain frequency as data is obtained or after a certain time period has passed, with the data being stored in a memory until transmittal (e.g., every minute, five minutes, or other predetermined time period). In certain other aspects, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain other aspects, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative aspects, the introducer can include a needle or similar sharp. As would be readily recognized by a person skilled in the art, other types of introducers, such as sheaths or blades, can be present in alternative aspects. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more aspects. After opening the access pathway, the needle or other introducer can be withdrawn so that it does not represent a sharps hazard. In certain aspects, suitable needles can be solid or hollow, beveled or non-beveled, and/or circular or non-circular in cross-section. In more particular aspects, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In certain aspects, a tip of the needle (while present) can be angled over the terminus of sensor 104, such that the needle penetrates a tissue first and opens an access pathway for sensor 104. In certain aspects, sensor 104 can reside within a lumen or groove of the needle, with the needle similarly opening an access pathway for sensor 104. In either case, the needle is subsequently withdrawn after facilitating sensor insertion.

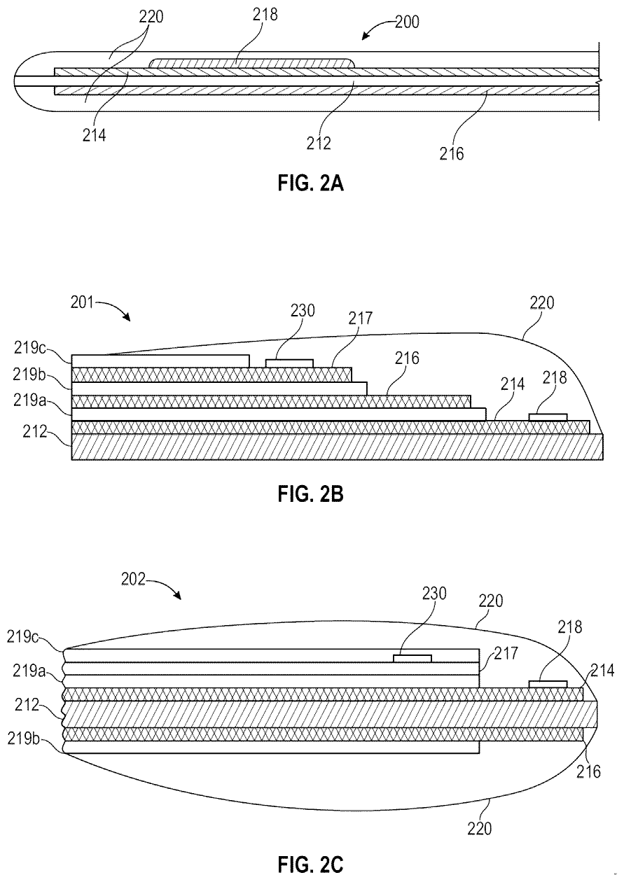

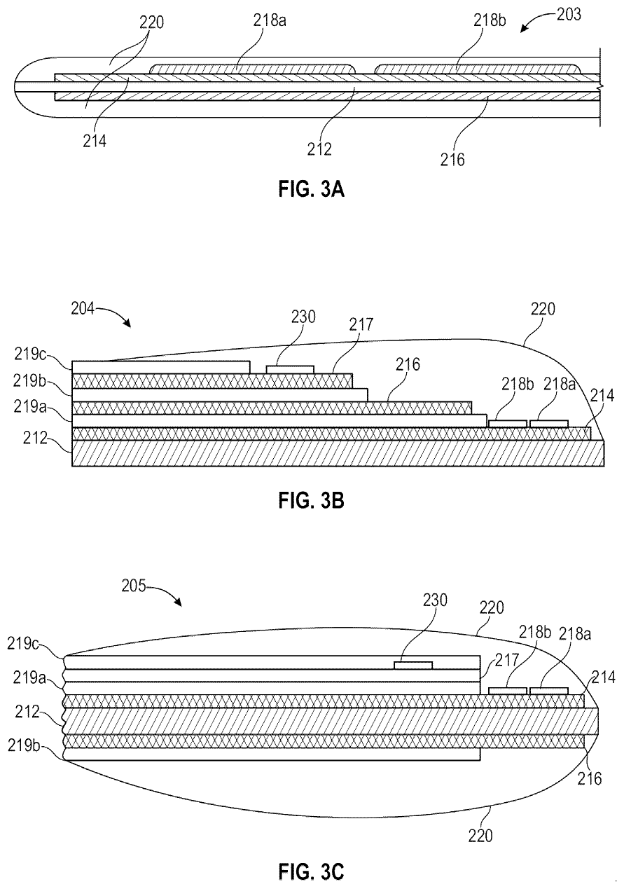

Sensor configurations featuring a single sensing layer that is configured for the detection of a corresponding single analyte can employ two-electrode or three-electrode detection motifs, as described further herein in reference to FIGS. 2A-2C. Sensor configurations featuring two different sensing layers for detection of separate analytes, either upon separate working electrodes or upon the same working electrode, are described separately thereafter in reference to FIGS. 3A-5C. Sensor configurations having multiple working electrodes can be particularly advantageous for incorporating two different sensing layers within the same sensor tail, since the signal contribution from each sensing layer can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape, substantially cylindrical in shape or any other suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Sensing layer 218 is disposed as at least one layer upon at least a portion of working electrode 214. Sensing layer 218 can include multiple spots or a single spot configured for detection of an analyte (e.g., creatinine), as discussed further herein.

Referring still to FIG. 2A, membrane 220 overcoats at least sensing layer 218. In certain aspects, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to sensing layer 218 (i.e., membrane 220 is a mass transport limiting membrane having some permeability for the analyte of interest). In some aspects, and further described below, membrane 220 is not crosslinked. Analyte sensor 200 can be operable for assaying an analyte (e.g., creatinine) by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in certain aspects, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 2B and 2C. As with sensor 200 shown in FIG. 2A, sensing layer 218 in analyte sensors 201 and 202 can include multiple spots or a single spot. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat sensing layer 218, as well as other sensor components, in analyte sensors 201 and 202, thereby serving as a mass transport limiting membrane. In certain aspects, the additional electrode 217 can be overcoated with membrane 220. Although FIGS. 2B and 2C have depicted electrodes 214, 216, and 217 as being overcoated with membrane 220, it is to be recognized that in certain aspects only working electrode 214 is overcoated. Moreover, the thickness of membrane 220 at each of electrodes 214, 216, and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the aspects disclosed herein, with alternative electrode and/or layer configurations remaining within the scope of the present disclosure.

FIG. 3A shows an illustrative configuration for sensor 203 having a single working electrode with two different sensing layers disposed thereon. FIG. 3A is similar to FIG. 2A, except for the presence of two sensing layers upon working electrode 214: first sensing layer 218a and second sensing layer 218b, which are responsive to different analytes and are laterally spaced apart from one another upon the surface of working electrode 214. Sensing layers 218a and 218b can include multiple spots or a single spot configured for detection of each analyte. The composition of membrane 220 can vary or be compositionally the same at sensing layers 218a and 218b. First sensing layer 218a and second sensing layer 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first sensing layer 218a and second sensing layer 218b disposed thereon. FIGS. 3B and 3C are otherwise similar to FIGS. 2B and 2C and can be better understood by reference thereto. As with FIG. 3A, the composition of membrane 220 can vary or be compositionally the same at sensing layers 218a and 218b.

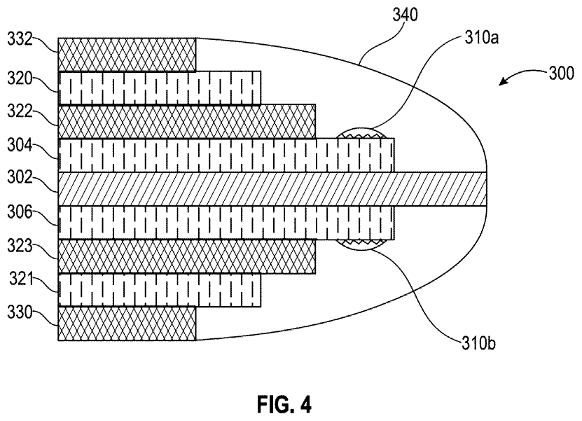

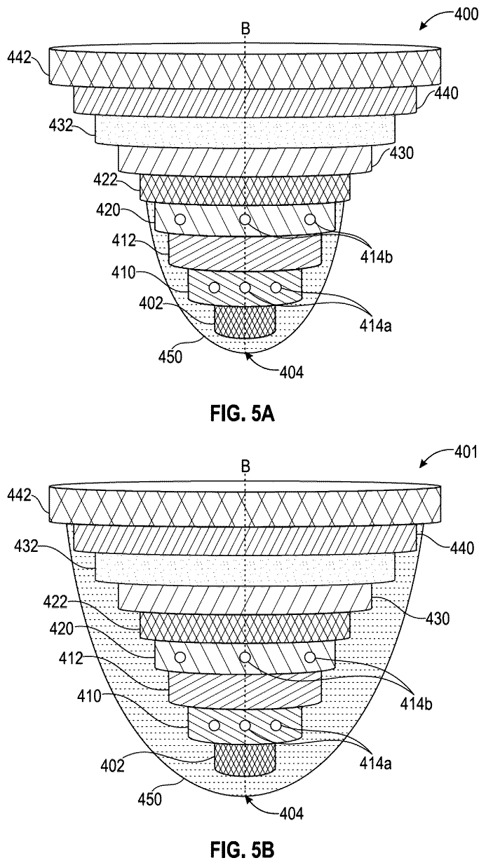

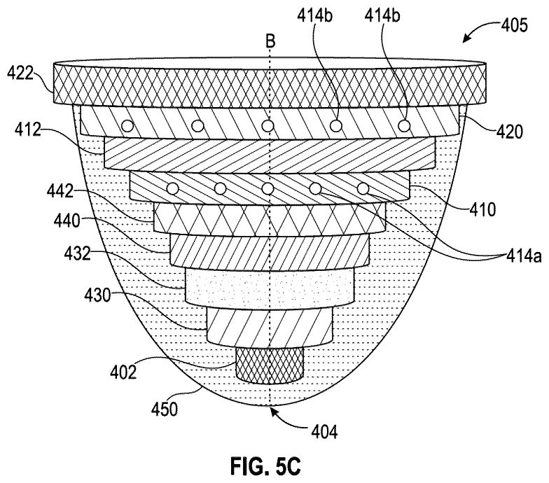

Illustrative sensor configurations having multiple working electrodes, specifically two working electrodes, are described in further detail in reference to FIGS. 4-5C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First sensing layer 310a is disposed upon the surface of working electrode 304, and second sensing layer 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least sensing layers 310a and 310b, according to various aspects, with other components of analyte sensor 300 or the entirety of analyte sensor 300 optionally being overcoated with membrane 340.

Like analyte sensors 200, 201, and 202, analyte sensor 300 can be operable for assaying an analyte (e.g., creatinine) by any of coulometric, amperometric, voltammetric, or potentiometric electrochemical detection techniques.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described hereinbelow. FIGS. 5A-5C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first sensing layers 414a and second sensing layers 414b, which are responsive to different analytes or the same analyte, are disposed upon the exposed surfaces of working electrodes 410 and 420, respectively, thereby allowing contact with a fluid to take place for sensing. Although sensing layers 414a and 414b have been depicted as three discrete spots in FIG. 5A, it is to be appreciated that fewer or greater than three spots, including a continuous layer of sensing layer, can be present in alternative sensor configurations.

In FIG. 5A, sensor 400 is partially coated with membrane 450 upon working electrodes 410 and 420 and sensing layers 414a and 414b disposed thereon. FIG. 5B shows an alternative sensor configuration in which the substantial entirety of sensor 401 is overcoated with membrane 450. Membrane 450 can be the same or vary compositionally at sensing layers 414a and 414b.

It is to be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of sensing layers 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

Several parts of the sensor are further described below.

The present disclosure relates to a creatinine sensor comprising
a first working electrode,
a creatinine sensing layer disposed on at least a portion of the first working electrode, the creatinine sensing layer comprising a redox mediator, creatinine amidohydrolase, creatine amidinohydrolase, and sarcosine oxidase, and
a first hydrophilic polyurethane membrane overcoating at least the creatinine sensing layer.

In the creatinine sensor, a working electrode (e.g., the first working electrode, the second working electrode) can be any suitable conductive material. Examples of suitable conductive materials include, e.g., aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements. In some aspects, a working electrode (e.g., the first working electrode, the second working electrode) can comprise carbon.

The creatinine sensing layer senses creatinine and is disposed on at least a portion of the first working electrode. The creatinine sensing layer can be continuously or discontinuously disposed on at least a portion of the working electrode. A discontinuous application means that the sensing layer can form a discrete shape on the working electrode, such as a spot, a line, or a plurality (e.g., an array) of spots and/or lines. The number of spots or lines is not considered to be particularly limited, but can range from 2 to about 10 (e.g., 2, 3, 4, 5, 6, 7, 8, 9, or 10, including about 3 to about 8, or from about 4 to about 6). In any of the aspects herein, the creatinine sensing layer can be continuous on the working electrode. In other aspects, the sensing layer can be discontinuous on the working electrode.

The total size of the creatinine sensing layer or layers (e.g., combined area of all spots, layers, or active areas) can be at least about 0.05 mm² and can be up to about 100 mm². In some aspects, the total size can be about 0.05 mm² to about 100 mm², about 0.05 mm² to about 75 mm², about 0.05 mm² to about 50 mm², about 0.05 mm² to about 40 mm², about 0.05 mm² to about 30 mm², about 0.05 mm² to about 25 mm², about 0.05 mm² to about 15 mm², about 0.05 mm² to about 10 mm², about 0.05 mm² to about 5 mm², about 0.05 mm² to about 1 mm², or about 0.05 mm² to about 0.1 mm². In a particular aspect, the total size of the sensing layer or layers ranges from about 0.05 to about 0.1 mm², about 0.05 to about 100 mm², about 0.1 to about 50 mm², about 0.5 to about 30 mm², about 1 to about 20 mm², or about 1 to about 15 mm².

The creatinine sensing layer or layers typically have a thickness that ranges from about 0.1-10 μm. For example, each creatinine sensing layer can be 0.1 μm thick or more (e.g., 0.2 μm or more, 0.3 μm or more, 0.5 μm or more, 0.8 μm or more, 1 μm or more, 2 μm or more, 3 μm or more, 5 μm or more, or 8 μm or more) and typically can have a thickness of 10 μm or less (e.g., 8 μm or less, 5 μm or less, 3 μm or less, 2 μm or less, 1 μm or less, 0.8 μm or less, 0.5 μm or less, 0.3 μm or less, or 0.2 μm or less). In an example, each sensing layer present can have a thickness of about 0.1 to about 10 μm, about 0.2 to about 8 μm, about 0.5 to about 5 μm, about 1 to about 4 μm, or about 2 μm.

In some aspects, a conductive material such as, for example, carbon nanotubes, graphene, or metal nanoparticles, can be combined within the sensing layer or layers to promote rapid attainment of a steady state current. Conductive material can be included in a range from about 0.1% to about 50% by weight (pbw) of the sensing layer (e.g., about 1 to about 50 pbw, about 1 to about 10 pbw, or about 0.1 to about 10 pbw).

The creatinine sensing layer comprises a redox mediator and three enzymes: creatinine amidohydrolase, creatine amidinohydrolase, and sarcosine oxidase. If necessary, one or more cofactors can be included with the enzymes, which serve as catalysts for the electron transfer. A suitable cofactor includes, e.g., nicotinamide adenine dinucleotide, in either oxidized (NAD) or reduced form (NADH). In some aspects, the creatinine sensing layer does not include an oxygen scavenger.

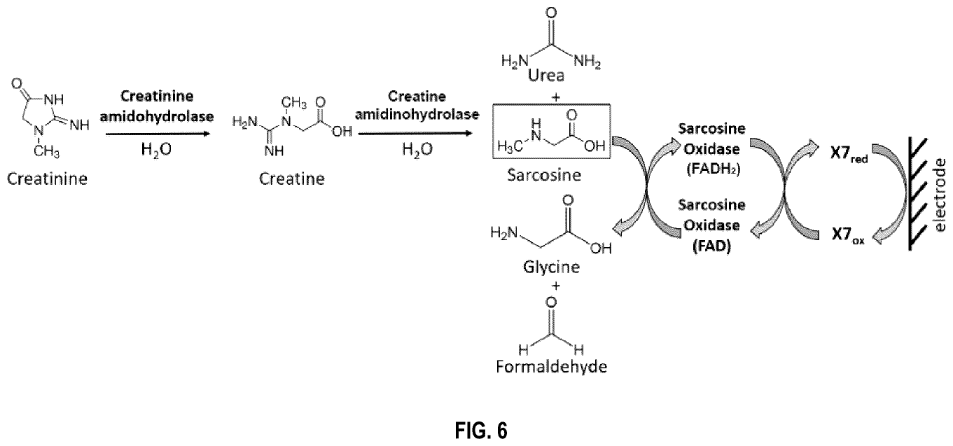

Without wishing to be bound by theory, it is believed that creatinine amidohydrolase catalyzes the hydrolysis of creatinine in a sample to creatine; that creatine amidinohydrolase catalyzes the oxidation of creatine to sarcosine with urea as a by-product; that sarcosine is converted into glycine and formaldehyde in the presence of sarcosine oxidase; and that sarcosine oxidase reduces the redox mediator which then transfers electrons to the working electrode, which in turn can then be oxidized at an anode, i.e., the working electrode. The electrons transferred during this reaction provide the basis for creatinine detection at the working electrode. The electrochemical signal obtained can then be correlated to the amount of creatinine that was initially present in the sample at the time of measurement. An illustrative scheme of how the enzymes interact with the creatinine analyte and the working electrode is shown in FIG. 6.

In addition to the enzymes, the creatinine sensing layer comprises a redox mediator. In some aspects, the redox mediator can comprise a polymer and an electron transfer agent.

In some aspects, the polymer in the redox mediator can be any suitable polymer that allows the transfer of electrons between the electron transfer agent and the working electrode. For example, the polymer can be a polyvinylpyridine (e.g., poly(4-vinylpyridine; PVP)), a polyvinylimidazole (e.g., poly(1-vinylimidazole; PVI)), poly(aniline), poly(pyrrole), poly(acetylene), poly(acrylic acid), styrene/maleic anhydride copolymer, methylvinylether/maleic anhydride copolymer, poly(vinylbenzylchloride), poly(allylamine), poly(lysine), poly(acrylamide-co-1-vinyl imidazole), poly(4-vinylpyridine) quaternized with carboxypentyl groups, or poly(sodium 4-styrene sulfonate). These polymers can be considered precursor polymers in that the polymers are further modified to immobilize (e.g., attach) the electron transfer complex. In some aspects, the polymer can comprise a backbone comprising poly(4-vinylpyridine), poly(1-vinylimidazole), poly(styrene), poly(thiophene), poly(aniline), poly(pyrrole), poly(acetylene), or any combination thereof. In other aspects, the polymer can comprise a polymer or copolymer repeat unit that can comprise at least one (e.g., 1, 2, 3, 4, 5, or 6) pendant pyridinyl group, imidazolyl group, or both a pyridinyl and imidazolyl group. For example, a suitable polymer can include partially or fully quaternized poly(4-vinylpyridine) and poly(1-vinylimidazole), in which quaternized pyridine and imidazole groups, respectively, can be used to form spacers by reaction with (e.g., complexation with) an electron transfer agent.

In some aspects, the electron transfer agent in the redox mediator can comprise a transition metal complex. The transition metal in the transition metal complex can be any suitable transition metal that can be effectively reduced and oxidized in the method described herein. For example, the transition metal complex can comprise osmium, ruthenium, iron, cobalt, vanadium, or a combination thereof. In some aspects, the transition metal can be ruthenium or osmium, particularly osmium. According to some aspects, suitable electron transfer agents can include low-potential osmium complexes, such as those described in U.S. Pat. Nos. 6,134,461, 6,605,200, 6,736,957, 7,501,053, and 7,754,093, the disclosures of each of which are incorporated herein by reference in their entirety. Other suitable examples of electron transfer mediators and polymer-bound electron transfer mediators can include those described in U.S. Pat. Nos. 8,444,834, 8,268,143, and 6,605,201, the disclosures of which are incorporated herein by reference in their entirety.

The transition metal complex can further comprise at least one ligand, which can be monodentate or multidentate (e.g., bidentate, tridentate, tetradentate). Typically, the complex will include enough ligands to provide a full coordination sphere. In some aspects, at least one ligand (e.g., 1, 2, 3, 4, 5, or 6) can comprise a nitrogen-containing heterocycle.

Monodentate ligands include, for example —F, —Cl, —Br, —I, —CN, —SCN, —OH, NH₃, alkylamine, dialkylamine, trialkylamine, alkoxy, a heterocyclic compound, compounds containing such groups, a solvent molecule (e.g., H₂O, EtOH), or a reactive group. For example, an alkyl (e.g., C₁₋₁₂, C₁₋₆, C₁₋₄, C₁₋₃) or aryl (e.g., phenyl, benzyl, naphthyl) portions of a ligand can be optionally substituted by F, Cl, Br, I, alkylamino, dialkylamino, trialkylammonium (except aryl portions), alkoxy, alkylthio, aril. Examples of suitable heterocyclic monodentate ligands include imidazole, pyrazole, oxazole, thiazole, pyridine, and pyrazine, each of which can be unsubstituted or substituted, as described herein (e.g., with at least one reactive group, such as 1, 2, 3, or 4 reactive groups).

Examples of suitable bidentate ligands include, for example, 1,10-phenanthroline, an amino acid, oxalic acid, acetylacetone, a diaminoalkane, an ortho-diaminoarene, 2,2′-biimidazole, 2,2′-bioxazole, 2,2′-bithiazole, 2-(2-pyridyl)imidazole, and 2,2′-bipyridine, each of which can be unsubstituted or substituted, as described herein (e.g., substituted with at least one reactive group, such as 1, 2, 3, or 4 reactive groups). Particularly suitable bidentate ligands for the electron transfer complex include substituted and unsubstituted 2,2′-biimidazole, 2-(2-pyridyl)imidazole, and 2,2′-bipyridine. Examples of suitable terdentate ligands include, for example, diethylenetriamine, 2,2′,2″-terpyridine, 2,6-bis(N-pyrazolyl)pyridine, each of which can substituted or unsubstituted (e.g., substituted with one more alkyl groups, such as methyl, or one or more reactive groups).

A suitable 2,2′-biimidazole ligand can be a ligand according to formula (I):

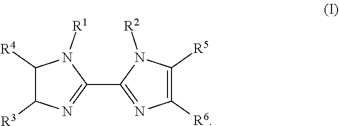

In formula (I), R¹ and R² are the same or different and each is a substituted or unsubstituted alkyl, alkenyl, or aryl. Generally, R¹ and R² are the same or different and each is an unsubstituted C₁₋₁₂ alkyl (e.g., C₁₋₄ alkyl). In some aspects, both R¹ and R² are methyl.

In formula (I), R³, R⁴, R⁵, and R⁶ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R³ and R⁴, in combination, or R⁵ and R⁶, in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R³, R⁴, R⁵, and R⁶ are the same or different and each is H or an unsubstituted C₁₋₁₂ alkyl (e.g., C₁₋₄ alkyl). In some aspects, R³, R⁴, R⁵, and R⁶ are all H.

A suitable 2-(2-pyridyl)imidazole ligand can be a ligand according to formula (II):

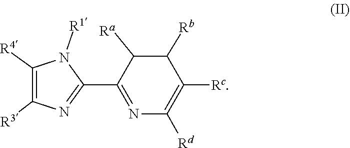

In formula (II), R^{3′}, R^{4′}, R^{a}, R^{b}, R^{c}, and R^{d} are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R^{3′} and R^{4′}, in combination, or two adjacent substituents of R^{a}, R^{b}, R^{c}, and R^{d} (e.g., R^{a} and R^{b}, R^{b} and R^{c}, or R^{c} and R^{d}) in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R^{3′}, R^{4′}, R^{a}, R^{b}, R^{c}, and R^{d} are the same or different and each is H or an unsubstituted C₁₋₁₂ alkyl (e.g., C₁₋₄ alkyl). In some aspects, R^{3′}, R^{4′}, R^{a}, R^{b}, R^{c}, and R^{d} are all H.

A suitable 2,2′-bipyridine ligand can be a ligand according to formula (III).

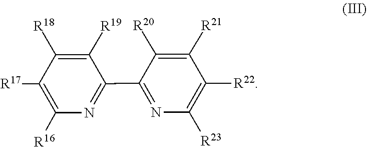

In formula (III), R¹⁶, R¹⁷, R¹⁸, R¹⁹, R²⁰, R²¹, R²², and R²³ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Typically, the alkyl and alkoxy portions are C-n. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H).

Specific examples of suitable combinations include R¹⁶ and R²³ are both H or both methyl and/or R¹⁷ and R²³ are both H or both methyl and/or R¹⁸ and R²¹ are both H or both methyl and/or R¹⁹ and R²⁰ are both H or both methyl. An alternative combination is where one or more adjacent pairs of substituents (e.g., R¹⁶ and R¹⁷, R¹⁷ and R¹⁸, R¹⁸ and R¹⁹, R²³ and R²², R²² and R²¹, or R²¹ and R²⁰), in combination, form a saturated or unsaturated 5- or 6-membered ring (e.g., benzo).

In an aspect, the one or more ligand is 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline), or a combination of any of these.

In some aspects, the transition metal complex can include a counterion (X) to balance the charge of the transition metal. Typically, there can be 1 to 5 (i.e., 1, 2, 3, 4, or 5) counterions. Multiple counterions in the complex are not necessarily all the same. Examples of suitable counterions include anions, such as halide (e.g., fluoride, chloride, bromide, or iodide), sulfate, phosphate, hexafluorophosphate, and tetrafluoroborate, and cations (e.g., a monovalent cation), such as lithium, sodium, potassium, tetralkylammonium, and ammonium. In some aspects, the counterion is a halide, such as chloride.

In an aspect, the transition metal complex can be an osmium transition metal complex that can comprise one or more ligands, wherein at least one (e.g., 1, 2, 3, 4, 5, or 6) ligand that can comprise a nitrogen-containing heterocycle (e.g., imidazole, pyrazole, oxazole, thiazole, pyridine, and pyrazine). In some aspects, the osmium transition metal complex can comprise one or more ligands selected from 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline).

In an aspect, the redox mediator can comprise an osmium complex bonded to a polymer or copolymer of poly(I-vinyl imidazole) or poly(4-vinylpyridine). The poly(4-vinylpyridine)-based polymer is a prepolymer that has been modified, as shown in the following structure, to attach an osmium complex (e.g., a poly(biimidizyl) osmium complex):

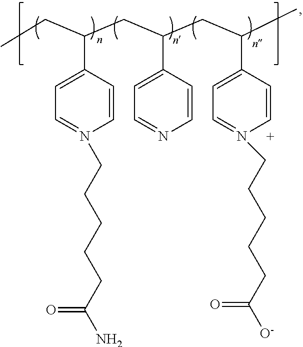

wherein n can be 2, n′ can be 17, and n″ can be 1. Other reactive groups and/or spacer groups can be used.

In an aspect, the electron redox mediator can comprise an osmium-containing poly(4-vinylpyridine)-based polymer, as shown below

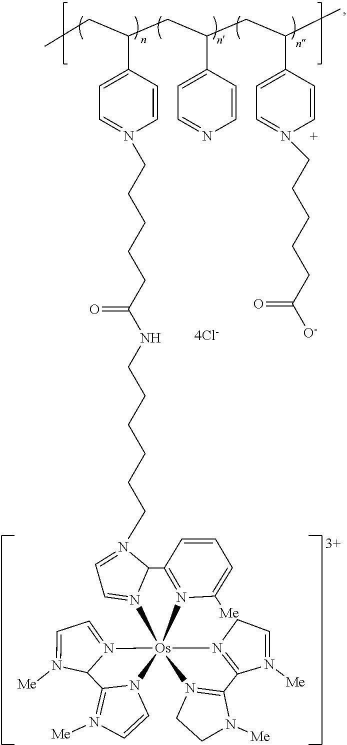

wherein n is 2, n′ is 17, and n″ is 1.

In some aspects, the electron transfer agent can be attached (e.g., non-leachably and/or covalently bonded) to the polymer in the redox mediator. For example, covalent bonding of the electron transfer agent to the polymer can take place by polymerizing a monomer unit bearing a covalently bound electron transfer agent, or the electron transfer agent can be reacted with the polymer separately after the polymer has already been synthesized.

According to some aspects, a bifunctional spacer can be used to attach (e.g., covalently bond) the electron transfer agent to the polymer in the redox mediator, with a first reactive group being reactive with the polymer (e.g., a functional group capable of quaternizing a pyridine nitrogen atom or an imidazole nitrogen atom) and a second reactive group being reactive with the electron transfer agent (e.g., a functional group that is reactive with a ligand coordinating a metal ion). Typically, covalent bonds are formed between the two reactive groups to generate a linkage. Suitable reactive groups include, for example, activated ester (e.g., succinimidyl, benzotriazolyl, or an aryl substituted with one more electron withdrawing groups, such as sulfo, nitro, cyano, or halo), acrylamido, acyl azido, acyl halide, carboxy (—COO— or —CO₂H), aldehyde, ketone, alkyl halide, alkyl sulfonato, anhydride, aziridino, epoxy, halotriazinyl, imido ester, isocyanato, isothiocyanato, maleimido, sulfonyl halide, amino, thiol (—SH), hydroxy, pyridinyl, imidazolyl, and hydroxyamino. The reaction between two reactive groups can form a covalent linkage between the transition metal complex and the polymer that is a carboxamido, thioether, hydrazonyl, oximyl, alkylamino, ester, carboxylic ester, imidazolium, pyridinium, ether, thioether, aminotriazinyl, triazinyl ether, amidinyl, urea, urethanyl, thiourea, thioether, sulfonamide, or any combination. In addition to the reactive groups, the bifunctional spacer typically can further comprise an alkylenyl (i.e., —(CH₂)ₙ—) and/or ethylenyloxy (i.e., —(CH₂CH₂O)ₙ—, in which n and m are each independently an integer from 1 to 12 (e.g., 1 to 11, 1 to 10, 1 to 9, 1 to 8, 1 to 7, 1 to 6, 1 to 5, 1 to 4, 1 to 3, or 1 to 2).

In some aspects, the creatinine sensing layer, and in particular, the redox mediator can further comprise a cross linking agent. In general, the cross linking agent is any suitable multifunctional (e.g., bifunctional) short chain molecule that enables the electron transfer agent to attach (e.g., covalently bond) to the polymer of the redox mediator. For example, the cross linking agent can be include a polyepoxide (e.g., a polyethylene glycol diglycidylether (PEGDGE), ethylene glycol diglycidyl ether (EGDGE), resorcinol diglycidyl ether, 1,2,7,8-diepoxyoctane, Gly3), cyanuric chloride, N-hydroxysuccinimide, an imidoester, epichlorohydrin, or a combination thereof. In an aspect, the cross linking agent is a polyethylene glycol diglycidylether (PEGDGE) of the following formula:

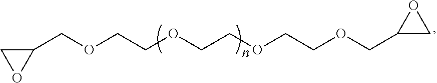

wherein n is an integer from 1 to about 50 (e.g., 1 to about 45, 1 to about 40, 1 to about 35, 1 to about 30, 1 to about 25, about 5 to about 50, about 5 to about 45, about 5 to about 40, about 5 to about 35, or about 5 to about 30).

In a particular example, the PEGDGE can be PEGDGE200, PEGDGE400 (n is 10), PEGDGE500, PEGDGE600, PEGDGE1000, or PEGDGE2000, in which the number denotes the average molecular weight (Mₙ). In an aspect, the crosslinking agent can be PEGDGE400.

The redox mediator can be applied to the working electrode using any suitable technique, such as spray coating, painting, inkjet printing, stenciling, roller coating, dip coating, or any combination thereof. In some aspects, the redox mediator can be applied by dip coating at least a portion of the working electrode into a solution of the redox mediator. One application or multiple applications can be applied (e.g., 1, 2, 3, 4, 5, 6, 7, 8, 9, or 10 applications). In some aspects, the redox mediator can be applied in 1, 2, 3, or 4 applications (e.g., passes). In some aspects, the redox mediator can be applied in 1 or 2 applications (e.g., passes).

In some aspects, one or more of the enzymes can be attached (e.g., covalently attached or unleachably bound) to the polymer portion of the redox mediator. In some aspects, one or more of the enzymes can be covalently bonded to the polymer portion of the redox mediator. Covalent bonding of one or more of the enzymes to the redox mediator (e.g., polymer) can take place via a crosslinking agent, as described herein, and a reactive site on the enzyme. Thus in such instances, an enzyme can be electronically “wired” to a working electrode through the redox mediator. In an aspect, a hydrogel can be formed upon crosslinking an enzyme and its wire on electrodes. In another aspect, at least a portion of an enzyme can diffuse into the polymer and/or hydrogel and become attached but not necessarily covalently bonded to the polymer.

In some aspects, the creatinine sensing layer can further comprise an albumin, which can act as an enzyme stabilizer. In an aspect, the albumin can be a serum albumin, such as bovine serum albumin or human serum albumin. In certain aspects, the sensing layer can comprise human serum albumin.

In certain aspects, the sensing layer can include a ratio of albumin stabilizer to enzyme (e.g., creatinine amidohydrolase) from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In certain aspects, the sensing layer can include a ratio of albumin stabilizer to enzyme from about 1:1 to about 1:10, e.g., from about 1:1 to about 1:9, from about 1:1 to about 1:8, from about 1:1 to about 1:7, from about 1:1 to about 1:6, from about 1:1 to about 1:5, from about 1:2 to about 1:9, from about 1:3 to about 1:8, from about 1:3 to about 1:7 or from about 1:4 to about 1:6.

In any of the aspects, the creatinine sensing layer can comprise a pH buffer. The buffer can be any suitable composition that is water soluble and controls (i.e., maintains) the pH of the sensing composition within a pH of about 5 to about 8 (e.g., maintains a pH of about 5, about 5.5, about 6, about 6.5, about 7, about 7.5, or about 8). In some aspects, the pH can be controlled to be within a range of about 6 to about 8. For example, the buffer can comprise a phosphate (e.g., monobasic and dibasic sodium phosphate), 4-(2-hydroxyethyl)piperazine-1-ethanesulfonic acid (HEPES), 2-(N-morpholino)ethanesulfonic acid (MES), 3-(N-morpholino) propanesulfonic acid (MOPS), 2-amino-2-(hydroxymethyl)-1,3-propanediol (TRIS), a carbonate (e.g., carbonic acid and a carbonate salt, such as sodium carbonate; sodium carbonate and sodium bicarbonate), or a citrate (e.g., citric acid and a citrate salt, such as trisodium citrate). The buffer can optionally comprise one or more (e.g., 1, 2, 3, or 4) additional salts (e.g., Group I or Group 11 halide salts, e.g., sodium chloride, potassium chloride, magnesium chloride). In an aspect, the buffer can be phosphate-buffered saline (PBS), which comprises disodium hydrogen phosphate, sodium chloride, and optionally potassium chloride and potassium dihydrogen phosphate. In another aspect, the buffer can be MES or a phosphate buffer, which can comprise phosphate, sodium chloride, potassium chloride, and/or magnesium chloride.

In some aspects, the buffer typically can be an aqueous buffer. In other aspects, non-aqueous solvents can be present, such as an alcohol (e.g., ethanol). In some aspects, the buffer can comprise water as the only solvent. In other aspects, the buffer can comprise water and at least one (e.g., 1, 2, or 3) non-aqueous solvents in any suitable ratio, such as a non-aqueous solvent to water volume ratio ranging from 99.9:0.1 to 0.1:99.9. In some aspects, the non-aqueous solvent to water volume ratio can be about 1:99, about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, or about 99:1, etc.). In a specific example, ethanol (EtOH) and water can be used in a volume ratio ranging from 50:50 to 90:10 EtOH:H₂O (e.g., 70:30, about 75:25, about 80:20, about 85:15, or about 90:10, etc.).

In some aspects, the first hydrophilic polyurethane membrane can be permeable to creatinine. It was unexpectedly discovered that in vivo sensor sensitivity to creatinine can be greatly improved by replacing a traditional poly(4-vinylpyridine)-based membrane with a hydrophilic polyurethane membrane, such as those disclosed elsewhere herein. In general, serum contains a low concentration of creatinine (e.g., about 100 μM to about 200 μM), such that a permeable hydrophilic polyurethane membrane can enable the detection of creatinine. In an aspect, the hydrophilic polyurethane membrane can have sufficient creatinine permeability to provide a creatinine sensitivity of about 1 nA/mM or greater when exposed to a sample comprising creatinine.

In some aspects, using a hydrophilic polyurethane membrane can result in reduced background interference, including ascorbate interference, particularly when compared to a poly(4-vinylpyridine)-based membrane (e.g., a crosslinked polyvinylpyridine-co-styrene polymer) and a perfluorinated sulfonated membrane, such as a tetrafluoroethylene-perfluoro-3,6-dioxa-4-methyl-7-octenesulfonic acid copolymer-based membrane (e.g., NAFION®, The Chemours Company, Wilmington, DE), both of which are crosslinked. In some aspects, the creatinine sensor does not include a poly(4-vinylpyridine)-based membrane (e.g., a crosslinked poly(4-vinylpyridine)-based membrane, such as a crosslinked copolymer of vinylpyridine and styrene). In some aspects, the creatinine sensor does not include a perfluorinated sulfonated membrane, such as a tetrafluoroethylene-perfluoro-3,6-dioxa-4-methyl-7-octenesulfonic acid copolymer-based membrane (e.g., a NAFION™ membrane).

Material and processing costs can be reduced with increasing performance in association with use of a hydrophilic polyurethane membrane over that of a traditional poly(4-vinylpyridine)-based membrane. In particular, a polyurethane membrane is much lower in price than a poly(4-vinylpyridine)-based membrane and is applied using a much simpler process (e.g., no cross-linker required, no curing required, and reduced processing time overall).

The hydrophilic polyurethane membrane can optionally coat all or part of a working electrode (e.g., the first working electrode, the second working electrode) and optionally any counter or reference electrode that can be present. In an aspect, the hydrophilic polyurethane membrane can coat (e.g., encapsulate) the entire creatinine sensor, including the working electrode(s) with the sensing layer(s), and any counter electrodes, reference electrodes, and/or substrates that can be present. In an aspect, the hydrophilic polyurethane membrane can coat (e.g., encapsulate) the sensor tail.

In some aspects, the hydrophilic polyurethane membrane can be formed from a hydrophilic polyurethane that can be aliphatic, aromatic, or both aliphatic and aromatic.

In some aspects, the hydrophilic polyurethane can comprise an aliphatic hydrophilic polyurethane comprising a reaction product of an aliphatic organic diisocyanate and a diol. The aliphatic organic diisocyanate can be any suitable aliphatic compound comprising two isocyanate groups (—O═C═N). Typically, the two isocyanate groups can be on the terminal ends of the aliphatic compound. The aliphatic compound can be cyclic or acyclic, linear or branched, and typically can have 2 to 14 carbons (e.g., 2 to 12, 2 to 10, 2 to 9, 2 to 8, 2 to 7, or 2 to 6 carbons), not including the diisocyanate groups.

In some aspects, the aliphatic compound can include one or more substituents other than the two diisocyanate groups. A substituted aliphatic compound typically comprises at least one substituent (e.g., 1, 2, 3, 4, 5, 6, etc.) in any suitable position (e.g., 1-, 2-, 3-, 4-, 5-, or 6-position, etc.). Suitable substituents include, e.g., halo, C₁₋₆ alkyl, C₁₋₆ haloalkyl, C₂₋₆ alkenyl, C₂₋₆ alkynyl, hydroxy, nitro, cyano, amino, mono- or di-C₁₋₆ alkylamino, C₁₋₆ alkoxy, C₆₋₁₀ aryloxy, aralkoxy, carboxy, C₁₋₆ alkylcarboxy, amido, C₁₋₆ alkylamido, C₁₋₆ haloalkylamido, C₆₋₁₀ aryl, heteroaryl, and heterocycloalkyl. In some aspects, the substituent can be 1 or 2 moieties selected from C₁₋₄ alkyl (e.g., methyl), halo, and/or haloalkyl.

In some aspects, the aliphatic organic diisocyanate can be selected from the group consisting of isophorone diisocyanate (IPDI), 1,4-cyclohexyl diisocyanate (CHDI), decane-1,10-diisocyanate, lysine diisocyanate (LDI), 1,4-butane diisocyanate (BDI), 1,5-pentanediisocyanate (PDI), hydrogenated xylene diisocyanate (HXDI), 2,2,4-trimethylhexamethylene diisocyanate (TMDI), hexamethylene diisocyanate (HDI), dicyclohexylmethane-4,4′-diisocyanate (H₁₂MDI), 1,3-bis(isocyanatemethyl)cyclohexane (BIMC), biuret, and combinations thereof.

In some aspects, the hydrophilic polyurethane can comprise an aromatic hydrophilic polyurethane comprising a reaction product of an aromatic organic diisocyanate and a diol. The aromatic organic diisocyanate can be any suitable aromatic compound comprising two isocyanate groups (—O═C═N). The aromatic compound can be any mono-, bi-, or tricyclic carbocyclic ring system having one, two, or three aromatic rings, for example, phenyl, naphthyl, anthracenyl, or biphenyl. In some aspects, the aromatic compound can contain from, for example, 6 to 30 carbon atoms, from 6 to 18 carbon atoms, from 6 to 14 carbon atoms, from 6 to 12 carbon atoms, or from 6 to 10 carbon atoms, not including the diisocyanate groups.

In some aspects, the aromatic compound can include one or more substituents other than the two diisocyanate groups. A substituted aromatic compound can typically comprise at least one substituent (e.g., 1, 2, 3, 4, 5, 6, etc.) in any suitable position (e.g., 1-, 2-, 3-, 4-, 5-, or 6-position, etc.). Suitable substituents include, e.g., halo, C₁₋₆ alkyl, C₁₋₆ haloalkyl, C₂₋₆ alkenyl, C₂₋₆ alkynyl, hydroxy, nitro, cyano, amino, mono- or di-C₁₋₆ alkylamino, C₁₋₆ alkoxy, C₆₋₁₀ aryloxy, aralkoxy, carboxy, C₁₋₆ alkylcarboxy, amido, C₁₋₆ alkylamido, C₁₋₆ haloalkylamido, C₆₋₁₀ aryl, heteroaryl, and heterocycloalkyl. In some aspects, the substituent can be 1 or 2 moieties selected from C₁₋₄ alkyl (e.g., methyl), halo, and/or haloalkyl.

In some aspects, the aromatic organic diisocyanate can be selected from the group consisting of methylene diphenyl diisocyanate (MDI), toluene diisocyanate (TDI), o-toluidine diisocyanate (TODI), 4,4′-diphenylmethane diisocyanate, p-phenylene diisocyanate (PPDI), xylene diisocyanate, hexamethylene diisocyanate, toluene diisocyanate, 1,5-naphthalene diisocyanate (NDI), polymeric MDI, and combinations thereof.

In some aspects, the diol can be selected from the group consisting of a polyether polyol, polylactide diol, polyglycolide diol, poly(lactide-b-glycolide), poly(lactide-co-caprolactone) diol, poly(hexamethylene carbonate) diol, polycarbonate diol, poly(ethylene terephthalate diol), poly(ethylene adipate) diol, poly(butylene adipate) diol, fatty acid-based linear diol, castor oil-based diol, soybean oil-based diol, ethylene glycol, diethylene glycol, propylene glycol, dipropylene glycol, 1,4-butanediol (BDO), 1,6-hexanediol (HDO), 1,3-butanediol, 2-methyl-1,3-propanediol, 1,5-pentanediol, neopentylglycol, 1,4-cyclohexanedimethanol (CHDM), 2,2-bis[4-(2-hydroxyethoxy) phenyl]propane (HEPP), hexamethylenediol, heptanediol, nonanediol, dodecanediol, 3-methyl-1,5-pentanediol, glycerine, sorbitol, sucrose, xylitol, pentaerythritol, ethylenediamine, butanediamine, hexamethylenediamine, hydroxyethyl resorcinol (HER), poly(tetramethylene ether) glycol (PTMG), poly(polytetrahydrofuran carbonate) diol, bis(2-hydroxyethyl)terephthalate (BHET), and a combination thereof.

In some aspects, the diol can be a polyether polyol, such as a hydroxyl-terminated polyether polyol derived from a diol or polyol having a total of from 2 to 15 carbon atoms (e.g., 2, 3, 4, 5, 6, 7, 8, 9, 10, I1, 12, 13, 14, or 15 carbon atoms). The polyether polyol can have any suitable number average molecular weight, such as about 100 g/mol or more to about 20,000 g/mol or less. In some aspects, the polyether polyol can have a low end of a range of a number average molecular weight of about 200 g/mol or more (e.g., about 300 g/mol or more, about 400 g/mol or more, about 500 g/mol or more, about 600 g/mol or more, about 700 g/mol or more, about 800 g/mol or more, about 900 g/mol or more, about 1000 g/mol or more, about 1200 g/mol or more, about 1400 g/mol or more, about 1600 g/mol or more, about 1800 g/mol or more, about 2000 g/mol or more, about 2200 g/mol or more, about 2400 g/mol or more, about 2600 g/mol or more, about 2800 g/mol or more, about 3000 g/mol or more, about 3200 g/mol or more, about 3400 g/mol or more, about 3600 g/mol or more, about 3800 g/mol or more, about 4000 g/mol or more, about 4200 g/mol or more, about 4400 g/mol or more, about 4600 g/mol or more, about 4800 g/mol or more, about 5000 g/mol or more, about 5200 g/mol or more, about 5400 g/mol or more, about 5600 g/mol or more, about 5800 g/mol or more, about 6000 g/mol or more, about 6200 g/mol or more, about 6400 g/mol or more, about 6600 g/mol or more, about 6800 g/mol or more, about 7000 g/mol or more, about 7200 g/mol or more, about 76 g/mol or more, about 7800 g/mol or more, about 8000 g/mol or more, about 8200 g/mol or more, about 8600 g/mol or more, about 8800 g/mol or more, about 9000 gmol or more, about 9200 g/mol or more, about 9400 g/mol or more, about 9600 g/mol or more, about 9800 g/mol or more, about 10,000 g/mol or more, about 14,000 g/mol or more, about 12,000 g/mol or more, about 13,000 g/mol or more, about 14,000 g/mol or more, about 15,000 g/mol or more, about 16,000 g/mol or more, about 18.000 g/mol or more, or about 19,000 g/mol or more). In some aspects of these lower end of the molecular weight range, the polyether polyol can have an upper end of the number average molecular weight range that is about 20,000 g/mol or less (e.g., about 19,000 g/mol or less, about 18,000 g/mol or less, about 17,000 g/mol or less, about 16,000 g/mol or less, about 15,000 g/mol or less, about 14,000 g/mol or less, about 13,000 g/mol or less, about 12,000 g/mol or less, about 11,000 g/mol or less, about 10,000 g/mol or less, about 9800 g/mol or less, about 9600 g/mol or less, about 9400 g/mol or less, about 9200 g/mol or less, about 9000 g/mol or less, about 8800 g/mol or less, about 8600 g/mol or less, about 8400 g/mol or less, about 8200 g/mol or less, about 8000 g/mol or less, about 7800 g/mol or less, about 7600 g/mol or less, about 7400 g/mol or less, about 7200 g/mol or less, about 7000 g/mol or less, about 6800 g/mol or less, about 6600 g/mol or less, about 6400 g/mol or less, about 6200 g/mol or less, about 6000 g/mol or less, about 5800 g/mol or less, about 5600 g/mol or less, about 5400 g/mol or less, about 5200 g/mol or less, about 5000 g/mol or less, about 4800 g/mol or less, about 4600 g/mol or less, about 4400 g/mol or less, about 4200 g/mol or less, about 4000 g/mol or less, about 3800 g/mol or less, about 3600 g/mol or less, about 3400 g/mol or less, about 3200 g/mol or less, about 3000 g/mol or less, about 2800 g/mol or less, about 2600 g/mol or less, about 2400 g/mol or less, about 2200 g/mol or less, about 2000 g/mol or less, about 1800 g/mol or less, about 1600 g/mol or less, about 1400 g/mol or less, about 1200 g/mol or less, about 1000 g/mol or less, about 900 g/mol or less, about 800 g/mol or less, about 700 g/mol or less, about 600 g/mol or less, about 500 g/mol or less, about 400 g/mol or less, about 300 g/mol or less, or about 200 g/mol or less). In some aspects, the polyol ether has a number average molecular weight of about 200 g/mol to about 15,000 g/mol or about 200 g/mol to about 5000 g/mol or about 400 g/mol to about 10,000 g/mol or about 400 g/mol to about 8000 g/mol or about 500 g/mol to about 5000 g/mol or about 600 g/mol to about 12,000 g/mol.

In some aspects, the diol can be a polyether polyol selected from the group consisting of polycaprolactone diol, poly(hydroxybutyrate) diol, poly(butyleneglycol) diol, polypropylene oxide diol, poly(ethylene glycol diol), poly(tetramethylene oxide diol), and a combination thereof.

In some aspects, the polyether polyol can be based on polyethylene oxide (i.e., polyethylene glycol (PEG)), polypropylene glycol (PPG), poly(trimethylene ether) glycol, or polytetrahydrofuran (PTMEG). For example, the polyether polyol can have the structure

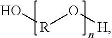

wherein R is 2, 3, or 4 carbons (e.g., a C₂-4 alkylene residue), and n is an integer from 1 to about 325. In an aspect, the polyether polyol can comprise PEG, PPG, or PTMEG with the following structures.

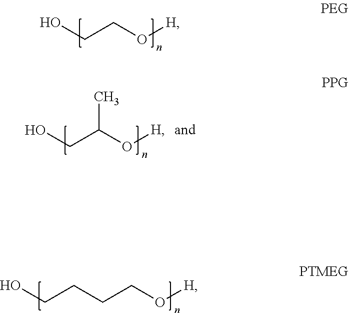

wherein n in each formula is an integer from 1 to 325 (e.g., 1 to 300, 1 to 250, 1 to 200, 1 to 150, 1 to 100, 1 to 80, 2 to 80, 5 to 80, 7 to 80, 7 to 70, 7 to 60, 7 to 50, 7 to 40, 7 to 30, etc.).

In some aspects, the polyether polyol can also include a polyamide adduct of an alkylene oxide, such as an ethylenediamine adduct comprising the reaction product of ethylenediamine and propylene oxide, a diethylenetriamine adduct comprising the reaction product of diethylenetriamine with propylene oxide, and similar polyamide type polyether polyols.

In some aspects, a chain extender can be used along with the diisocyanate and diol to form the polyurethane. The chain extender can comprise a diol (e.g., 2 hydroxy groups) or polyol (e.g., at least 3 hydroxy groups), each comprising 2 to 20 carbon atoms (e.g., 2, 3, 4, 5, 6, 7, 8, 9, 10, 11, 12, 13, 14, 15, 16, 18, 19, or 20 carbon atoms). In some aspect, the diol or polyol can comprise 2 to 12 carbon atoms or 2 to 10 carbon atoms. In some aspects, the polyol can have 2, 3, or 4 carbons per hydroxy group.

The chain extender typically can have a molecular weight (including a number average molecular weight for polymeric structures) that is about 3,000 g/mol or less (e.g., about 2800 g/mol or less, about 2600 g/mol or less, about 2400 g/mol or less, about 2200 g/mol or less, about 2000 g/mol or less, about 1800 g/mol or less, about 1600 g/mol or less, about 1400 g/mol or less, about 1200 g/mol or less, about 1000 g/mol or less, about 900 g/mol or less, about 800 g/mol or less, about 700 g/mol or less, about 600 g/mol or less, about 500 g/mol or less, about 400 g/mol or less, about 300 g/mol or less, or about 200 g/mol or less) to about 60 g/mol or more (e.g., about 70 g/mol or more, about 80 g/mol or more about 90 g/mol or more, or about 100 g/mol or more). In some aspects, the chain extender can have a molecular weight that ranges from about 60 g/mol to about 2,000 g/mol.

In some aspects, the chain extender can be selected from the group consisting of ethylene glycol, diethylene glycol, propylene glycol, dipropylene glycol, 1,4-butanediol (BDO), 1,6-hexanediol (HDO), 1,3-butanediol, 2-methyl-1,3-propanediol, 1,5-pentanediol, neopentylglycol, 1,4-cyclohexanedimethanol (CHDM), 2,2-bis[4-(2-hydroxyethoxy) phenyl]propane (HEPP), hexamethylenediol, heptanediol, nonanediol, dodecanediol, 3-methyl-1,5-pentanediol, glycerine, sorbitol, sucrose, xylitol, pentaerythritol, ethylenediamine, butanediamine, hexamethylenediamine, hydroxyethyl resorcinol (HER), poly(tetramethylene ether) glycol (PTMG), poly(polytetrahydrofuran carbonate) diol, bis(2-hydroxyethyl)terephthalate (BHET), and combinations thereof.

In some aspects, the hydrophilic polyurethane can comprise an aliphatic hydrophilic polyurethane. In some aspects, the hydrophilic polyurethane can comprise an aromatic hydrophilic polyurethane. In some aspects, the hydrophilic polyurethane can comprise both an aliphatic and aromatic hydrophilic polyurethane.

Any suitable method can be used to produce the hydrophilic polyurethane by reacting the diisocyanate and diol, including reacting a diisocyanate and a polyether polyol optionally in the presence of a chain extender. For example, in one aspect, the process is a so-called “one-shot” process in which all three reactants are added to an extruder reactor and reacted. The equivalent weight amount of the diisocyanate to the total equivalent weight amount of the hydroxyl containing components, that is, the diol (e.g., polyether polyol) and optional chain extender, can be from about 0.95 to about 1.10, or from about 0.96 to about 1.02, or from about 0.97 to about 1.005. The reaction temperature is not particularly limited but can be from about 175-245° C.

In another aspect, the hydrophilic polyurethane can also be prepared using a pre-polymer process, in which the diol (e.g., polyol) is reacted with generally an equivalent excess of one or more diisocyanates to form a pre-polymer solution having free or unreacted diisocyanate therein. In this method, the reaction can generally be carried out at temperatures of from about 80-220° C. Subsequently, a chain extender can be added in an equivalent amount generally equal to the number of unreacted isocyanate end groups as well as to any free or unreacted diisocyanate compounds. The overall equivalent ratio of the total diisocyanate to the total equivalent weight amount of the diol (e.g., polyol) intermediate and the chain extender is thus from about 0.95 to about 1.10, or from about 0.96 to about 1.02, or from about 0.97 to about 1.05. The chain extension reaction temperature is generally from about 180-250° C.

If necessary, a polymerization catalyst can be used to prepare the polyurethane. Generally, any conventional catalyst can be utilized to react the diisocyanate with the diol (e.g., polyether polyol) and/or chain extender. Examples of suitable catalysts include a tertiary amine (e.g. triethylamine, dimethylcyclohexylamine, N-methylmorpholine, N,N′-dimethylpiperazine, 2-(dimethylaminoethoxy)ethanol, diazabicyclo[2.2.2]octane), an organometallic compound (e.g., a titanic ester, an iron compound (e.g. ferric acetylacetonate), or a tin compound (e.g. stannous diacetate, stannous dioctoate, stannous dilaurate, dibutyltin diacetate, dibutyltin dilaurate), or any combination thereof.

In some aspects, the hydrophilic polyurethane can be a thermoplastic polyurethane elastomer. In such aspects, the hydrophilic polyurethane typically can be linearly segmented block copolymers comprising hard and soft segments. In some aspects, hard or soft segments can be pendant to the polymer backbone. In some aspects, the reaction between diisocyanates with chain extenders (e.g., short chain diols) will form hard (rigid) segments, and the reaction between diisocyanates with polyether polyol (e.g., long chain diols) will form soft (flexible) segments. In some aspects, the block copolymer can comprise soft segments comprising urethane and hard segments comprising urethane-diisocyanate. The ratio of hard to soft segments can be varied as needed.

In some aspects, the hydrophilic polyurethane can have a hardness that ranges from medium hard to hard. The Shore A hardness is a measure of the resistance of a material to indentation, and the scale ranges from 0 (extremely soft) to 100 (extremely hard). For example, and in some aspects, the hydrophilic polyurethane can have a Shore A hardness of at least about 60A, as measured in accordance with ASTM D2240 (last updated Jul. 23, 2021) using a durometer (e.g., using a type A indenter). The upper limit of the hardness of the hydrophilic polyurethane is not particularly limited. In some aspects, the hydrophilic polyurethane can have a Shore A hardness of about 60A to about 100A (e.g., about 60A to about 95A, about 60A to about 93A, about 60A to about 90A, about 60A to about 85A, about 65A to about 80A, about 70A to about 100A, about 70A to about 93A, about 70A to about 90A, about 70A to about 85A, about 70A to about 80A, about 75A to about 100A, about 75A to about 93A, about 75A to about 90A, about 75A to about 85A, about 75A to about 80A, about 60A, about 65A, about 70A, about 75A, about 80A, about 85A, about 90A, about 93A, about 95A, or about 100A). In some aspects, the hydrophilic polyurethane can have a Shore A hardness of at most about 93A, including about 80A, as measured in accordance with ASTM D2240 (last updated Jul. 23, 2021) using a durometer.

In some aspects, the hydrophilic polyurethane can have a hardness in accordance with the Shore D scale, as measured in accordance with ASTM D2240 (last updated Jul. 23, 2021) using a durometer (e.g., using a type D indenter). In some aspects, Shore D can be the preferred scale for harder materials. Thus, in some aspects, the hydrophilic polyurethane can have a Shore D hardness of about OD to about 100D (e.g., about 10D to about 100D, about 15D to about 100D, about 20D to about 100D, about 25D to about 100D, about 30D to about 100D, about 35D to about 100D, or about 40D to about 100D).

In some aspects, the hydrophilic polyurethane can have a hardness measureable only on the Shore A scale, measureable on both the Shore A and shore D scales, or measureable only on the Shore D scale.

In some aspects, the hydrophilic polyurethane is capable of absorbing about 5%-25% by weight of water. In some aspects, the hydrophilic polyurethane is capable of absorbing about 10%-25% (e.g., about 12%-25%, about 15%-25%, about 18%-25%, about 20%-25%, about 5%, about 8%, about 10%, about 12%, about 15%, about 18%, about 20%, about 21%, about 22%, about 23%, about 24%, or about 25%) by weight of water.

Examples of a suitable hydrophilic polyurethane include those commercially available under such tradenames as Hydrothane (AdvanSource Biomaterials Corporation, Wilmington, MA), e.g., Hydrothane AL 25-80A, which exhibits a water absorption rate of about 25%.

In some aspects, the hydrophilic polyurethane can have a Shore A hardness of about 80A and a water absorption rate of about 25%.

In some aspects, the hydrophilic polyurethane membrane is not crosslinked.

The coating of the hydrophilic polyurethane membrane over at least the creatinine sensing layer and optionally a background sensing layer can be performed using any suitable technique. In some aspects, the hydrophilic polyurethane membrane can be coated by spray coating, painting, inkjet printing, roller coating, dip coating, or any combination thereof. In an aspect, the coating comprises dipping the creatinine sensor comprising the creatinine sensing layer (e.g., the sensor tail) into a solution comprising the hydrophilic polyurethane and a solvent to provide a dipped creatinine sensor. The coating step can be performed once or multiple times (e.g., 2, 3, 4, 5, 6, 7, 8, 9, or 10 times), which will affect the thickness of the membrane coating. In an aspect, the coating step can be performed twice to form a bilayer. In an aspect, the coating step can be a dip coating. In an aspect, the coating step can be a dip coating performed 3 to 5 (i.e., 3, 4, or 5) times.

The hydrophilic polyurethane membrane typically has a thickness that ranges from about 1 μm to about 100 μm. For example, in some aspects, the membrane can have a thickness of about 1 μm or more (e.g., about 5 μm or more, about 10 μm or more, about 15 μm or more, about 20 μm or more, about 25 μm or more, about 30 μm or more, about 35 μm or more, about 40 μm or more, about 50 μm or more, about 60 μm or more, about 70 μm or more, about 80 μm or more, or about 90 μm or more) and typically can have a thickness of about 100 μm or less (e.g., about 90 μm or less, about 80 μm or less, about 70 μm or less, about 60 μm or less, about 50 μm or less, about 45 μm or less, about 40 μm or less, about 35 μm or less, about 30 μm or less, about 25 μm or less, about 20 μm or less, about 15 μm or less, about 10 μm or less, or about 5 μm or less). In an example, the membrane can have a thickness of about 5 to about 80 μm, about 10 to about 60 μm, about 20 to about 50 μm, about 20 to about 40 μm, about 25 to about 35 μm, or a thickness of about 30 μm.

In some aspects, the hydrophilic polyurethane membrane can comprise multiple layers in which each layer has a different composition. In an example, the membrane coating can be a bilayer membrane that can comprise a first layer that can comprise an aliphatic hydrophilic polyurethane and a second layer that can comprise an aromatic hydrophilic polyurethane. The variation in composition between layers allows tuning the permeability to creatinine.

In some aspects, the creatinine sensor can further be contained within a sensor housing that is configured for adherence to tissue (e.g., skin). If necessary, the sensor housing can include an adhesive layer that enables adhesion to the desired tissue. The sensor housing can hold all necessary components of the sensor, such as circuitry and a power source for operating the sensor. In some aspects, the power source (e.g., a coin cell battery) and/or active circuitry are not contained within the sensor housing. A processor can be communicatively coupled to the sensor, in which the processor is physically located within the sensor housing or a reader device. The power source can include one or more batteries, which can be rechargeable or single-use disposable batteries. Power management circuitry can regulate battery charging and power supply monitoring, boost power, or perform direct current (DC) conversions.

In some aspects, the creatinine sensor further comprises a sensor tail (e.g., insertion tip) configured for penetrating (e.g., implantation into) tissue. The sensor tail includes at least one working electrode and a creatinine sensing layer. A counter electrode can be present in combination with the at least one working electrode. The various electrodes can be at least partially stacked (layered) upon one another and/or laterally spaced apart from one another upon the sensor tail. In general, the sensor tail can be of sufficient size and shape to be positionable below the surface of the tissue (e.g., penetrating through the skin (dermis)) and into the subcutaneous space and in contact with the wearer's biofluid, such as interstitial fluid. Suitable sensor configurations can be substantially flat in shape, substantially cylindrical in shape, or any other suitable shape. In an example, a sensor tail can be about 5 mm in length, about 0.6 mm in width, and about 0.25 mm in thickness. Suitable tissues include, for example, skin, including the dermal layer, an interstitial layer, and/or a subcutaneous layer of the skin. In any of the sensor configurations disclosed herein, the various electrodes can be electrically isolated from one another by a dielectric material or similar insulator.

In addition to a working electrode, a sensing layer disposed on at least a portion of the working electrode, and a hydrophilic polyurethane membrane overcoating at least the creatinine sensing layer, the creatinine sensor further can comprise a reference electrode, a counter electrode, or both a reference electrode and a counter electrode in some aspects. In an aspect, the counter electrode can be carbon (e.g., screen-printed carbon), and the reference electrode can be Ag/AgCl. In a two electrode example, a working electrode and a second electrode that functions as both a counter electrode and reference electrode (i.e., a counter/reference electrode) can be used.

In some aspects, the creatinine sensor can comprise at least one insulation (e.g., dielectric) layer. In some aspects, the insulation layer can be comprised of a suitable dielectric material that can form a solid. In an example, the insulation layer can be formed from porcelain (ceramic), mica, glass, barium strontium titanate, a plastic (e.g., polystyrene, polytetrafluoroethylene, polyethylene terephthalate, polyethylene, polypropylene, polymethylmethacrylate, polysulfone, polydimethylsiloxane, polyvinyl chloride, or a combination thereof), or a metal oxide (e.g., silica, alumina, titania, zirconia, tantalum oxide, etc.).

In some aspects, the creatinine sensor can comprise a substrate, wherein the working electrode can be disposed on the substrate. The substrate can be formed from any suitable inert material. In some aspects, the substrate can be biocompatible. Examples of a suitable substrate include titanium, a carbon-based substrate (e.g., cellulose, polylactic acid) and a plastic substrate (e.g., polyethylene terephthalate, polyethylene, polypropylene, polymethylmethacrylate, polysulfone, polydimethylsiloxane, polyvinyl chloride, etc.). In some aspects, the substrate can be disposed between the working electrode and a counter and/or reference electrode.

The creatinine sensor can be part of a system that can comprise a working electrode, a creatinine sensing layer disposed on at least a portion of the working electrode, and a circuit configured to connect and disconnect with the working electrode. In an aspect, the system can be a creatinine sensor (e.g., an enzymatic biosensor) comprising a working electrode, a creatinine sensing layer disposed on at least a portion of the working electrode, and a hydrophilic polyurethane membrane comprising an aliphatic and/or aromatic polyurethane overcoating at least the creatinine sensing layer. The creatinine sensing layer can comprise creatinine amidohydrolase, creatine amidinohydrolase, and sarcosine oxidase, one or more optional cofactors, and an osmium-containing poly(4-vinylpyridine)-based polymer as the redox mediator.

In any of the aspects herein, the creatinine sensor can comprise a background sensing electrode to detect background interference that can be subtracted from the creatinine signal to improve creatinine sensitivity. In some aspects, the background sensing electrode can comprise
a second working electrode,
a background sensing layer that does not detect creatinine disposed on at least a portion of the second working electrode, the background sensing layer comprising a redox mediator, optionally creatine amidinohydrolase, and optionally sarcosine oxidase, and
a second hydrophilic polyurethane membrane overcoating at least the background sensing layer,
wherein the background sensing layer does not comprise creatinine amidohydrolase.

The second working electrode can be the same or different than the first working electrode, as described herein. In some aspects, the second working electrode can be the same as the first working electrode.

The redox mediator in the background sensing layer can be the same or different from the redox mediator in the creatinine sensing layer, as described herein. In some aspects, the redox mediator in the background sensing layer can be the same as the redox mediator in the creatinine sensing layer.

The second hydrophilic polyurethane membrane can be the same or different than the first hydrophilic polyurethane membrane, as described herein. In some aspects, the second hydrophilic polyurethane membrane can be the same as the first hydrophilic polyurethane membrane.

Because the background sensing layer does not comprise creatinine amidohydrolase, the background sensing layer does not detect creatinine but rather detects background interference. In some aspects, the background sensing layer can comprise creatine amidinohydrolase and sarcosine oxidase. In some aspects, the background sensing layer does not comprise either creatine amidinohydrolase or sarcosine oxidase. In some aspects, the background sensing layer can comprise creatine amidinohydrolase but does not comprise sarcosine oxidase. In some aspects, the background sensing layer can comprise sarcosine oxidase but does not comprise creatine amidinohydrolase. In any of these aspects, any enzyme that is absent from the background sensing layer can be replaced with an albumin, such as human serum albumin, as described herein.

The present disclosure further relates to a method for sensing creatinine in a sample, such as a biofluid.

In some aspects, the method can comprise exposing a creatinine sensor described herein to a fluid (e.g., a biofluid) comprising creatinine; applying a potential to the first working electrode; obtaining a first signal that is proportional to a concentration of creatinine and background interference in the fluid (e.g., a biofluid); and correlating the first signal to the concentration of creatinine in the fluid (e.g., a biofluid).

In some aspects in which a background sensing electrode is present in the creatinine sensor, the method for sensing creatinine can comprise exposing a creatinine sensor as described herein to a fluid (e.g., a biofluid) comprising creatinine; applying a potential to the first working electrode and second working electrode; obtaining a first signal from the first working electrode that is proportional to a concentration of creatinine and background interference in the fluid (e.g., a biofluid); obtaining a second signal from the second working electrode that is proportional to a concentration of background interference in the fluid (e.g., a biofluid); and determining the concentration of creatinine in the fluid (e.g., a biofluid) correlated by subtracting the second signal from the first signal. In some aspects, creatinine concentration (mM) can be determined by the formula:

(creatinine signal (nA)−blank signal (nA))/creatinine response (nA/mM).

Known creatinine concentrations can be added to a control sample to determine a creatinine response value (nA/mM) for a sensor.

The sensing of the analyte (A) relies on having an oxidoreductase enzyme (AOx) electrically “wired” to the working electrode of the sensor through the redox mediator. During amperometric sensing, the electrode is poised at a potential (voltage) so that the analyte is reacted at a constant rate, which is proportional to the analyte concentration. In some aspects, in a creatinine sensor used herein, the potential (voltage) sufficient to drive the redox reaction and reduce background interference is less than +40 mV (e.g., less than +30 mV, less than +20 mV, less than +10 mV, less than +5 mV, less than 0 mV, less than −5 mV, less than −10 mV, less than −20 mV, less than −30 mV, less than −40 mV, less than −50 mV, less than −60 mV, less than −70 mV, less than −80 mV, less than −90 mV, less than −100 mV, or less than −110 mV) versus Ag/AgCl. For an analyte oxidation reaction (A to A+), the electrons will flow from the analyte (A) to the analyte-specific enzyme (AOx) to the redox mediator (e.g., Os³⁺) to the working electrode at a constant rate, producing a steady-state current. The reduced form of the redox mediator will be oxidized, resulting in a current spike. The current will then decay back to the original amperometric current as the redox system reaches steady-state once again.

It was surprisingly discovered that lowering the sensing potential reduced the interaction between the sensing electrode and electroactive interferents, which in turn decreased interference signal. In some aspects, the potential applied to the creatinine sensor can be less than +40 mV to about −125 mV, such as +20 mV to about −125 mV, about +5 mV to about −125 mV, about −5 mV to about −100 mV, about −10 mV to about-90 mV, or about −20 mV to about −80 mV, each relative to a Ag/AgCl reference. In some aspects, the potential applied can be about +35 mV, about +30 mV, about +20 mV, about +10 mV, about +5 mV, about −5 mV, about −10 mV, about −20 mV, about −30 mV, about −40 mV, about −50 mV, about −60 mV, about −70 mV, about −80 mV, about −90 mV, about −100 mV, about −110 mV, or about −120 mV, each relative to a Ag/AgCl reference. In some particular aspects, the potential applied can be about −80 mV vs Ag/AgCl.

In an example of measuring creatinine, electrode contacts are positioned on a first portion of the sensor situated above the skin surface and extend to a location in sensor tail. The first working electrode, a reference electrode, and a counter electrode are at a second portion of the sensor, typically at a bottom portion of the sensor tail. The first working electrode will comprise a creatinine sensing layer for detecting creatinine. The sensor can optionally comprise a second working electrode, a reference electrode, and a counter electrode at a third portion of the sensor, also typically at the bottom portion of the sensor tail. The second working electrode can comprise a background sensing layer for detecting background interferents, as described herein.

The present disclosure is also directed to a method for sensing creatinine comprising:
exposing a creatinine sensor (or system) as described herein, to a biofluid comprising creatinine;
wherein the creatinine sensor comprises a sensor tail comprising a first working electrode with a creatinine sensing layer disposed upon a surface of the first working electrode, and optionally a second working electrode with a background sensing layer disposed upon a surface of the second working electrode, and a hydrophilic polyurethane membrane (i) formed from a polyurethane that is aliphatic, aromatic, or both aliphatic and aromatic and (ii) having a first portion overcoating the creatinine sensing layer and an optional second portion overcoating the background sensing layer;
wherein the creatinine sensing layer comprises creatinine amidohydrolase, creatine amidinohydrolase, sarcosine oxidase, and the optional background sensing layer does not comprise creatinine amidohydrolase and can comprise albumin, optionally creatine amidinohydrolase, and optionally sarcosine oxidase, and
applying a potential (e.g., less than +40 mV, about +5 mV to about −125 mV, or about −80 mV, each relative to Ag/AgCl) to the first working electrode and optionally the second working electrode;
obtaining a first signal that is proportional to a concentration of creatinine and background interference in the biofluid;
optionally obtaining a second signal that is proportional to a concentration of background interferents in the biofluid; and either
correlating the first signal to the concentration of creatinine in the fluid or
determining the concentration of creatinine in the fluid by subtracting the second signal from the first signal.

In some aspects, the first signal and the second signal are measured at different times. In some aspects, the first signal and the second signal are obtained simultaneously via a first channel and a second channel.

In some aspects, the creatinine sensor is exposed to the biofluid in vivo. In general, the method uses a system (e.g., a creatinine sensor), as disclosed herein, for measuring a concentration of creatinine and can be used in an in vivo monitoring system, which while positioned in vivo in a user (e.g., a patient, such as a human) makes contact with the biofluid of the user and senses creatinine contained therein. An in vivo monitoring system can include one or more reader devices that receives sensed analyte data from a sensor control device. The reader device can process and/or display the sensed analyte data or sensor data in any number of forms to the user. In some aspects, the reader device can be a mobile communication device, such as a dedicated reader device (configured for communication with a sensor control device) optionally in conjunction with a computer system, a mobile telephone (e.g., a WiFi or internet-enabled smart phone), a tablet, a personal digital assistant (PDA), or a mobile smart wearable electronics assembly (e.g., a smart glass, smart glasses, watch, bracelet, or necklace). Configuring a reader device to an in vivo monitoring system is described at, for example, U.S. Pat. No. 11,371,957, the disclosure of which is incorporated herein by reference in its entirety.

The reader device typically includes an input component, a display, and processing circuitry, which can include one or more processors, microprocessors, controllers, and/or microcontrollers, each of which can be a discrete chip or distributed amongst (and a portion of) a number of different chips. The processing circuitry can include a communications processor having on-board memory and an applications processor having on-board memory. The reader device can further include radio frequency (RF) communication circuitry coupled with an RF antenna, a memory, multifunctional circuitry with one or more associated antennas, a power supply, power management circuitry, and/or a clock. It will be recognized that other hardware and functionality can be included in the reader device.

In some aspects, the creatinine sensor can provide an improved sensor accuracy by about 3-fold or higher (e.g., about 4-fold or higher, about 5-fold or higher, about 6-fold or higher, about 7-fold or higher, about 8-fold or higher, about 9-fold or higher, or about 10-fold or higher) compared to a creatinine sensor that does not include one, two, or all three of the following features: a hydrophilic polyurethane membrane as described herein, a sensing potential less than +40 mV vs Ag/AgCl, and background subtraction.

In some aspects, the creatinine sensor can provide an accurate (e.g., within about 20% of actual, within about 18% of actual, within about 15% of actual, within about 12% of actual, within about 10% of actual, within about 8% of actual, within about 5% of actual, within about 4% of actual, within about 3% of actual, within about 2% of actual, or within about 1% of actual) creatinine measurement. In some aspects, the creatinine sensor can provide a creatinine measurement within about 10% of actual (e.g., a control). In any of these aspects, the creatinine sensor can provide an accurate creatinine measurement over a period of one day or more (e.g., 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more, 16 days or more, 17 days or more, 18 days or more, 19 days or more, 20 days or more, or 21 days or more). In some aspects, the creatinine sensor can provide an accurate creatinine measurement over a period of 7 days or more. In some aspects, the creatinine sensor can provide an accurate creatinine measurement over a period of 14 days or more. In some aspects, the creatinine sensor can provide an accurate creatinine measurement over a period of 21 days or more.

### EXAMPLES

The example presented below is provided for the purpose of illustration only and the aspects described herein should in no way be construed as being limited to this example. Rather, the aspects should be construed to encompass any and all variations which become evident as a result of the teaching provided herein.

### Example 1

To determine the relationship between the amount of redox mediator and background interference, two different blank sensors that were laser cut (330 μm wide) with one working electrode (blank (background) sensing)) were prepared in accordance with Table 1. The sensing chemistry was applied with a dispense pattern of 3×18 drops using 2- or 4-passes with drying steps between passes. Because creatinine amidohydrolase is not present and replaced with bovine serum albumin (BSA), only the interference signal is detected without a creatinine signal.

Each sensor was dip-coated using 5 dips at 5 mm/s in a 60 mg/mL solution of HYDROTHANE™ AL25-80A (AdvanSource Biomaterial, Wilmington, MA) in 1:1 ethanol:Me-THF (Me-THF: methyl-tetrahydrofuran). AL25-80A is an aliphatic polyurethane with a water absorbance capacity of about 25 wt % and a hardness of about 80 Shore A. The drop volume was 0.6 nL, so 1 pass with a 3×18 pattern had a total volume of 32.4 nL. Sensors were dispensed with multiple passes and allowed to dry between each pass. The total volume of dispensed material will be proportional to the number of passes. For example, two 3×18 passes will have a total dispense volume of 64.8 nL.

TABLE 1

| Blank | Final (mg/ml) |
|---|---|
| Os-PVP polymer | 30.00 |
| Creatine amidinohydrolase | 21.00 |
| Bovine serum albumin | 10.50 |
| PEGDGE400 | 4.00 |
| SOx | 6.87 |
| In 10 mM MES with 3 mM KCl, pH 5.5 | |

Os-PVP polymer: osmium-containing poly(4-vinylpyridine)-based polymer
PEGDGE400: polyethylene glycol diglycidylether with an average molecular weight (Mn) of about 400 g/mol
SOx: sarcosine oxidase
MES: 2-(N-morpholino)ethanesulfonic acid

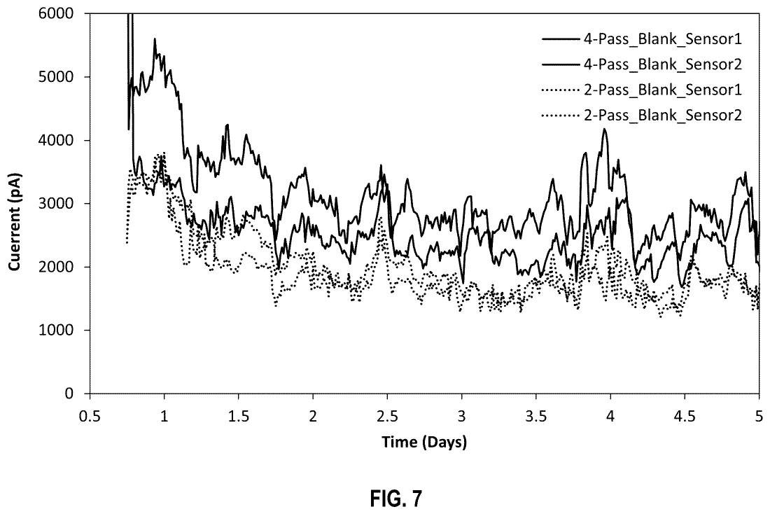

Each sensor was in vivo tested at +40 mV sensing potential with human serum, and the results are provided in FIG. 7. As seen in FIG. 7, adjusting the sensing chemistry deposition to have fewer passes provided less redox mediator (e.g., an osmium-containing poly(4-vinylpyridine)-based polymer crosslinked with PEGDGE400), which in turn reduced background signal in vivo.

### Example 2

To determine the relationship between the type of membrane and background interference, three creatinine sensors that were laser cut (330 μm wide) with one working electrode (creatinine sensing) were prepared with the sensing chemistry set forth in Table 2 and the membrane coating set forth in Table 3. The sensing chemistry was applied with a dispense pattern of 3×18 drops using 4-passes. The drop volume was 0.6 nL, so 4 passes with a 3×18 pattern had a total volume of 129.6 nL.

TABLE 2

| Creatinine | Final (mg/ml) |
|---|---|
| Os-PVP polymer | 30.00 |
| Creatine amidinohydrolase | 21.00 |
| Creatinine amidohydrolase | 10.50 |
| PEGDGE400 | 4.00 |
| SOx | 6.87 |
| In 10 mM MES with 3 mM KCl, pH 5.5 | |

Os-PVP polymer: osmium-containing poly(4-vinylpyridine)-based polymer
PEGDGE400: polyethylene glycol diglycidylether with an average molecular weight (Mn) of about 400 g/mol
SOx: sarcosine oxidase
MES: 2-(N-morpholino)ethanesulfonic acid

TABLE 3

| | | Dip-coating |
|---|---|---|
| Type | Polymer Solution | Steps |
| HydroThane | 60 mg/mL HydroThane AL25-80A in 1:1 | 5 dips at 5 mm/s |
| (polyurethane) | ethanol:MeTHF | |
| PVP | 4 mL 80 mg/mL PVP (160 kDa) in 80/20 | 4 dips at 1 mm/s |
| | EtOH/10 mM HEPES + 0.188 mL 100 mg/mL | |
| | PEGDGE400 | |
| PVP + Nafion | 1) 4 mL 80 mg/mL PVP (160 kDa) in 80/20 | 4 dips at 1 mm/s |
| | EtOH/10 mM HEPES + 0.188 mL 100 mg/mL | |
| | PEGDGE400 | |
| | 2) 100 mg/mL Nafion in EtOH | 3 dips at 5 mm/s |

PVP: poly(4-vinylpyridine)
HEPES: 4-(2-hydroxyethyl)piperazine-1-ethanesulfonic acid
Nafion: NAFION ™, The Chemours Company, Wilmington, DE

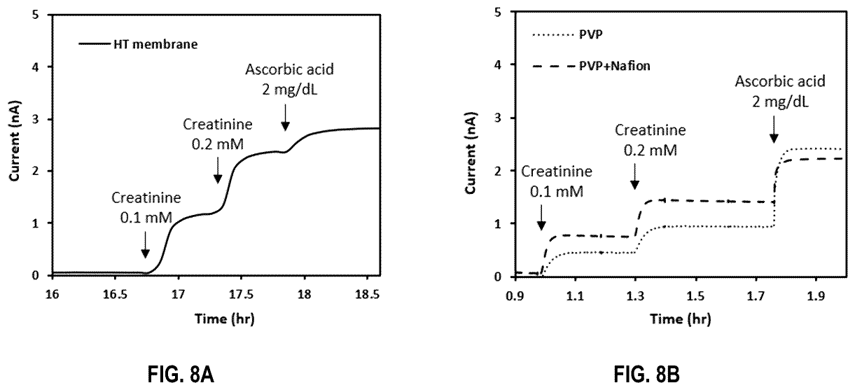

Sensors were tested in human serum containing a basal concentration of creatinine and using standard amperometry at +40 mV vs Ag/AgCl. At various time intervals, either 100 μM aliquots of creatinine or 2 mg/dL ascorbic acid were added. Time and current were recorded at a 5 minute data acquisition (DAQ) rate using a multichannel potentiostat (FIGS. 8A and 8B).

The graphs show the improved creatinine sensitivity of the sensor with the hydrophilic polyurethane membrane (FIG. 8A) compared to the sensors with the PVP and PVP+NAFION™ membranes (FIG. 8B). Setting the background interference to 100% for the PVP-containing sensor, the background interference was measured, as set forth in Table 4.

TABLE 4

| Sensor | Background Interference (%) |
|---|---|
| PVP | 100 |
| PVP + NAFION ™ | 44.5 |
| HYDROTHANE ™ AL25-80A | 18.7 |
| (hydrophilic polyurethane) | |

As seen in FIG. 8B and Table 4, sensors with PVP or PVP/NAFION™ membranes had lower creatinine sensitivity and significantly higher ascorbate interference. As seen in FIG. 8A and Table 3, the sensor with the hydrophilic polyurethane membrane had higher creatinine sensitivity and significantly lower ascorbate interference.

### Example 3

To determine the relationship between the sensing potential and background interference, four creatinine sensors that were laser cut (400 μm wide) with two working electrodes (creatinine sensing and blank (background) sensing) were prepared with the sensing chemistry set forth in Table 5, the dispensing information of Table 6, and the membrane coating set forth in Table 7. The sensing chemistry was applied with a dispense pattern of 5×10 drops. The drop volume was 0.6 nL, so 1 pass with a 5×10 pattern had a total volume of 30 nL. Total volume of dispensed material will be proportional to the number of passes. For example, two 5×10 passes will have a total dispense volume of 60 nL.

TABLE 5

| Sensing layer | | Stock | Mix | Final |
|---|---|---|---|---|
| chemistry | Component | (mg/ml) | (μl) | (mg/ml) |
| SL1_NoSOx | Os-PVP polymer | 160 | 116.25 | 30.49 |
| | PEGDGE400 | 160 | 15.25 | 4.00 |
| | 10 mM MES pH 5.5 with 3 | | 478.5 | |
| | mM KCl | | | |
| SOx | SOx | 189 | 50 | 12.5 |
| | 10 mM MES pH 5.5 with 3 | | 100.60 | |
| | mM KCl | | | |
| SL2 | HSA | 160 | 126.75 | 20.93 |
| | Creatine amidinohydrolase | 160 | 253.5 | 41.87 |
| | Creatinine amidohydrolase | 160 | 126.75 | 20.93 |
| | PEGDGE400 | 160 | 21.55 | 3.56 |
| | PVI (pH 7.4, 80 mg/ml) | 80 | 82.29 | 6.80 |
| | 10 mM PBS pH 7.4 with 3 | | 357.92 | |
| | mM KCl | | | |
| SL2_Blank | HSA | 160 | 253.5 | 41.87 |
| | Creatine amidinohydrolase | 160 | 253.5 | 41.87 |
| | PEGDGE400 | 160 | 21.55 | 3.56 |
| | PVI (80 mg/ml) | 80 | 82.29 | 6.80 |
| | 10 mM PBS pH 7.4 with 3 | | 357.92 | |
| | mM KCl | | | |

Os-PVP polymer: osmium-containing poly(4-vinylpyridine)-based polymer
PEGDGE400: polyethylene glycol diglycidylether with an average molecular weight (Mn) of about 400 g/mol
MES: 2-(N-morpholino)ethanesulfonic acid
SOx: sarcosine oxidase
HSA: human serum albumin
PVI: poly(1-vinylimidazole)

TABLE 6

| | |
|---|---|
| Creatinine Chemistry | 1) SL1_NoSOx - 1 pass |
| | 2) SOx- 1 pass |
| | 3) SL2 - 2 passes |
| Blank Chemistry | 1) SL1_NoSOx - 1 pass |
| | 2) SOx - 1 pass |
| | 3) SL2_Blank - 2 passes |

TABLE 7

| Polymer Solution | Dip-coating Steps |
|---|---|
| 75 mg/mL HydroThane AL25-80A | 5 dips at 5 mm/s |
| in 1:1 ethanol:MeTHF | |

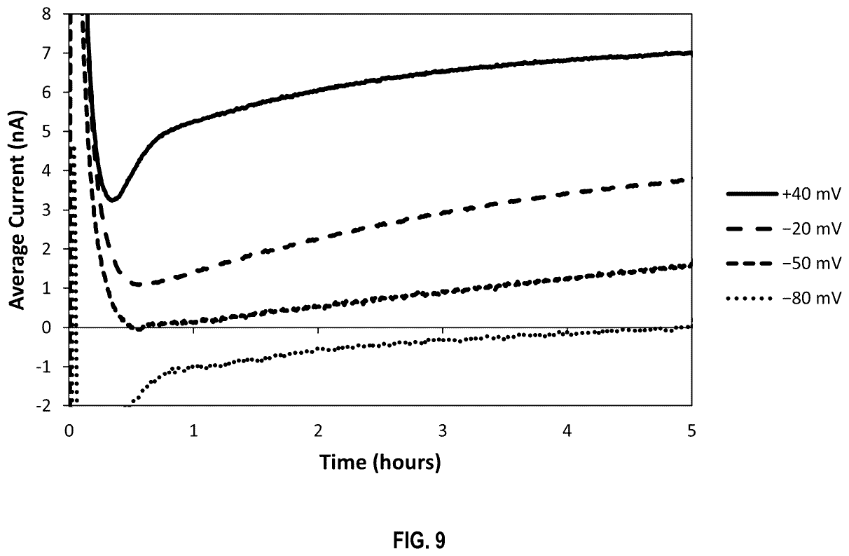

Sensors (n=3 or 4 for each type of sensor) were tested in human serum containing a basal concentration of creatinine and using standard amperometry at either +40 mV, −20 mV, −50 mV, or −80 mV vs Ag/AgCl. Time (hours) and current were recorded at a 5 minute data acquisition (DAQ) rate using a multichannel potentiostat (FIG. 9).

As seen in FIG. 9, it was surprisingly observed that lowering the sensing potential reduced the interaction between the sensing electrode and electroactive interferents, which in turn dramatically decreased the interference signal. At +40 mV, the background signal stabilized at >6 nA, whereas at −80 mV, the background signal stabilized at <1 nA.

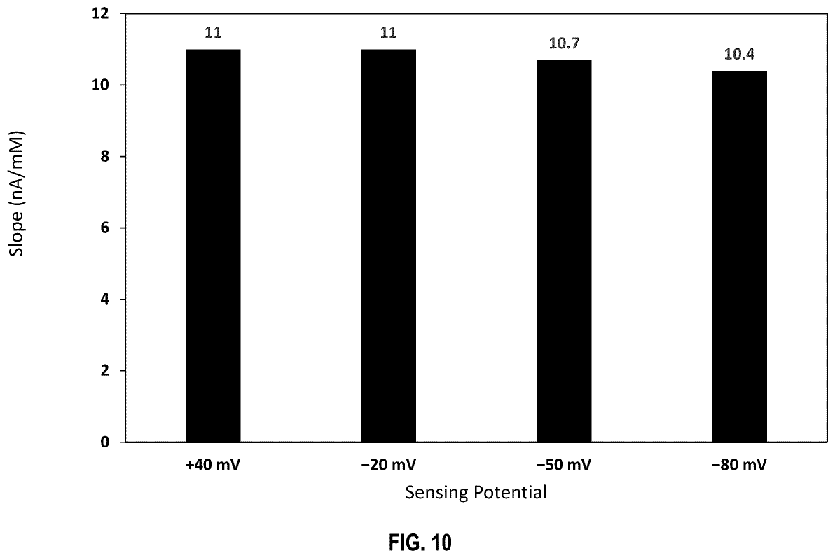

As seen in FIG. 10, decreasing signal potential reduced the background signal without compromising response as only about a 5% decrease in response was observed.

### Example 4

To determine the improvement in the accuracy of creatinine measurement, a creatinine sensor that was laser cut (400 μm wide) with two working electrodes (creatinine sensing and blank (background) sensing) was prepared in accordance with Tables 5-7.

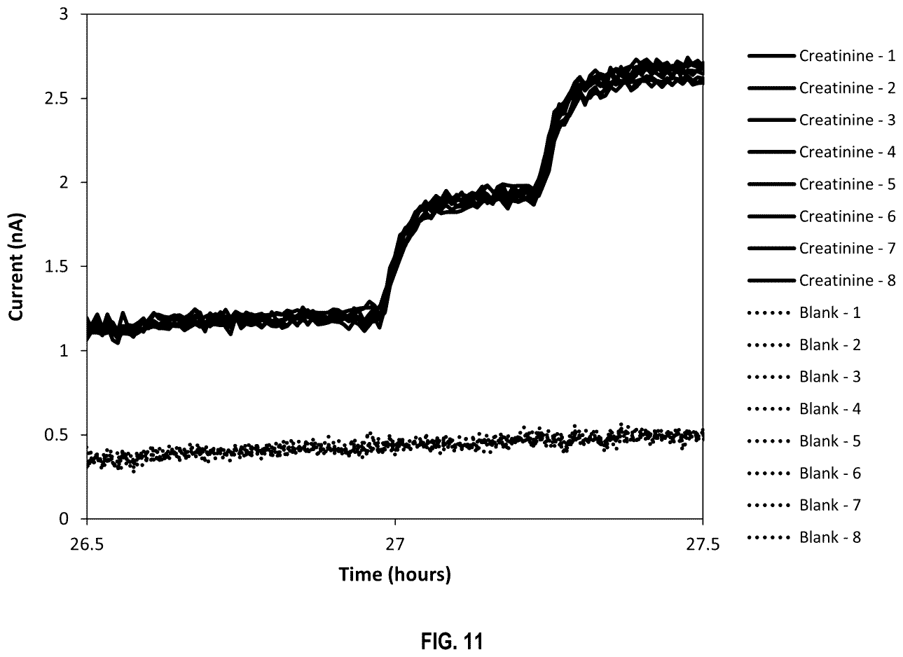

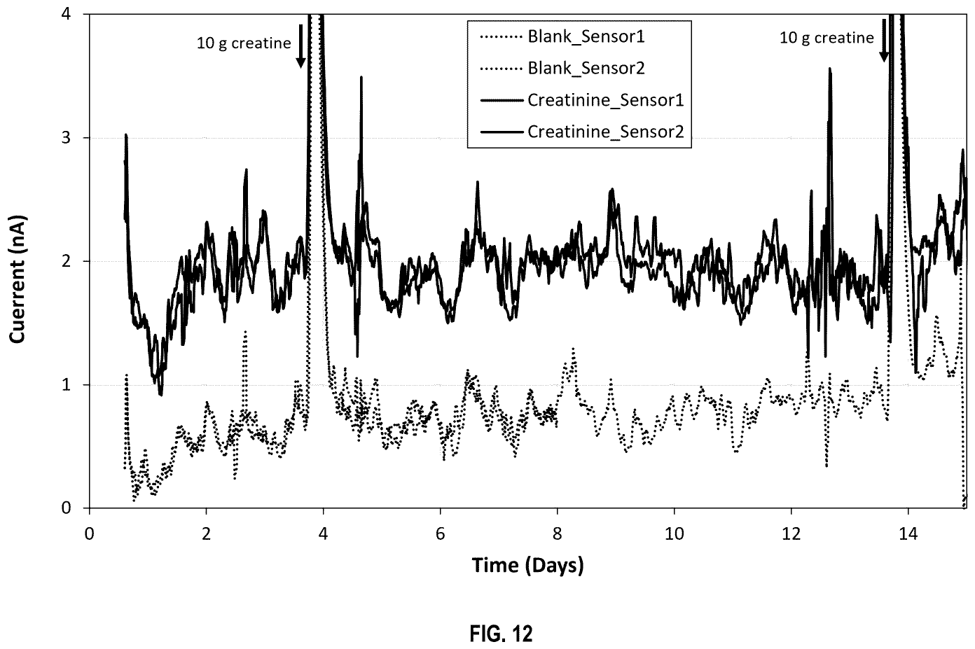

Sensors (n=8) were tested in human serum containing a basal concentration of creatinine and using standard amperometry at −80 mV vs Ag/AgCl. At various time intervals, 0.1 mM aliquots of creatinine were added. Time and current were recorded at a 5 minute data acquisition (DAQ) rate using a multichannel potentiostat (FIG. 11). FIG. 12 shows that in a dual channel sensor, blank (background) and creatinine channels drift up and down over time due to background interference, but the difference between the channels remains relatively constant (i.e., the channels have similar interference profiles), thereby indicating a stable creatinine signal.

As seen in FIG. 11, the creatinine channel showed a higher initial signal than the blank channel as a result of creatinine naturally present in serum (concentration: about 0.097 mM). Known creatinine concentrations were added to the serum to determine a creatinine response value (7.22 nA/mM). Initial channel signals were used to predict creatinine concentrations in the serum (with and without background subtraction), and the results are set forth in Table 8.

TABLE 8

| | |
|---|---|
| Without | Predicted [creatinine] = creatinine signal/creatinine |
| background | response = 1.21 nA/7.22 nA/mM = 0.168 mM |
| subtraction | |
| With | Predicted [creatinine] = (creatinine signal − blank |
| background | signal)/creatinine response = (1.21 nA − 0.43 nA)/ |
| subtraction | 7.22 nA/mM = 0.107 mM |
| Serum clinical | Actual [creatinine] = 0.097 mM |
| lab results | |

As seen in Table 8, there was a greater than 6-fold reduction in error in the creatinine measurement using background subtraction.

The creatinine sensitivity as a function of sensing potential and background subtraction is summarized in Table 9.

TABLE 9

| Sensing | Background | Predicted/actual [creatinine] in |
|---|---|---|
| Potential | Subtraction? | serum (97 μM creatinine) |
| +40 mV | No | >800% |
| −80 mV | No | 172% |
| −80 mV | Yes | 110% |

As seen in Table 9, reducing the sensing potential from +40 mV to −80 mV reduced background interference by more than 5-fold and dramatically improved sensor accuracy. However, the creatinine concentration was still overestimated. Adding in background subtraction to account for the remaining background interference, brought the predicted creatinine concentration in a human serum sample within 10% of the actual, clinical lab value.

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A creatinine sensor comprising:
a first portion configured to be positioned above a user's skin and a second portion configured to be positioned below the user's skin and in contact with the user's biological fluid to monitor the level of creatinine in vivo, the second portion comprising:
a first working electrode,
a creatinine sensing layer disposed on at least a portion of the first working electrode, the creatinine sensing layer comprising a redox mediator, creatinine amidohydrolase, creatine amidinohydrolase, and sarcosine oxidase, and
a first hydrophilic polyurethane membrane overcoating at least the creatinine sensing layer.

2. The creatinine sensor of claim 1, wherein the first hydrophilic polyurethane membrane is permeable to creatinine.

3. The creatinine sensor of claim 1, wherein the first hydrophilic polyurethane membrane is a thermoplastic polyurethane elastomer.

4. The creatinine sensor of claim 1, wherein the first hydrophilic polyurethane has a Shore A hardness of about 60A to about 93A.

5. (canceled)

6. The creatinine sensor of claim 1, wherein the first hydrophilic polyurethane is capable of absorbing about 5% to about 25% by weight water.

7. The creatinine sensor of claim 1, wherein the first hydrophilic polyurethane membrane is not crosslinked.

8. (canceled)

9. (canceled)

10. The creatinine sensor of claim 1, wherein the sensor further comprises a reference electrode, a counter electrode, or both a reference electrode and a counter electrode.

11. (canceled)

12. (canceled)

13. The creatinine sensor of claim 1, wherein the creatinine amidohydrolase, creatine amidinohydrolase, sarcosine oxidase, or any combination thereof is attached to the redox mediator.

14. The creatinine sensor of claim 1, wherein the redox mediator comprises a polymer and an electron transfer agent.

15. The creatinine sensor of claim 14, wherein the polymer comprises a backbone comprising poly(4-vinylpyridine), poly(1-vinylimidazole), poly(styrene), poly(thiophene), poly(aniline), poly(pyrrole), poly(acetylene), or any combination thereof.

16. The creatinine sensor of claim 14, wherein the polymer comprises a polymer or copolymer repeat unit comprising at least one pendant pyridinyl group, at least one pendant imidazolyl group, or both at least one pendant pyridinyl and at least one pendant imidazolyl group.

17. The creatinine sensor of claim 14, wherein the electron transfer agent comprises a transition metal complex.

18. The creatinine sensor of claim 17, wherein the transition metal complex comprises osmium, ruthenium, iron, cobalt, or any combination thereof.

19. The creatinine sensor of claim 17, wherein the transition metal complex is an osmium transition metal complex comprising one or more ligands, wherein at least one ligand comprises a nitrogen-containing heterocycle.

20. The creatinine sensor of claim 1, wherein the redox mediator comprises an osmium complex bonded to a poly(vinylpyridine)-based polymer.

21. The creatinine sensor of claim 14, wherein the polymer is crosslinked with a crosslinking agent.

22. The creatinine sensor of claim 21, wherein the crosslinking agent is a polyepoxide, cyanuric chloride, N-hydroxysuccinimide, an imidoester, epichlorohydrin, or any combination thereof.

23. The creatinine sensor of claim 21, wherein the crosslinking agent is a polyethylene glycol diglycidylether (PEGDGE).

24. The creatinine sensor of claim 1, wherein the creatinine sensing layer is continuously disposed on the first working electrode.

25. The creatinine sensor of claim 1, wherein the creatinine sensing layer is discontinuously disposed on the first working electrode.

26. The creatinine sensor of claim 1, wherein the second portion further comprises a background sensing electrode comprising
a second working electrode,
a background sensing layer that does not detect creatinine disposed on at least a portion of the second working electrode, the background sensing layer comprising the redox mediator, optionally creatine amidinohydrolase, and optionally sarcosine oxidase, and
a second hydrophilic polyurethane membrane overcoating at least the background sensing layer,
wherein the background sensing layer does not comprise creatinine amidohydrolase.

27. The creatinine sensor of claim 26, wherein the second hydrophilic polyurethane membrane is a thermoplastic polyurethane elastomer.

28. The creatinine sensor of claim 26, wherein the second hydrophilic polyurethane has a Shore A hardness of about 60A to about 93A.

29. (canceled)

30. The creatinine sensor of claim 26, wherein the second hydrophilic polyurethane is capable of absorbing about 5% to about 25% by weight water.

31. The creatinine sensor of claim 26, wherein the second hydrophilic polyurethane membrane is not crosslinked.

32. A method for sensing creatinine comprising:
exposing the creatinine sensor of claim 1 to a fluid comprising creatinine;
applying a potential to the first working electrode;
obtaining a first signal that is proportional to a concentration of creatinine and background interference in the fluid; and
correlating the first signal to the concentration of creatinine in the fluid.

33. A method for sensing creatinine comprising:
exposing the creatinine sensor of claim 26 to a fluid comprising creatinine;
applying a potential to the first working electrode and second working electrode;
obtaining a first signal from the first working electrode that is proportional to a concentration of creatinine and background interference in the fluid;
obtaining a second signal from the second working electrode that is proportional to a concentration of background interference in the fluid; and
determining the concentration of creatinine in the fluid by subtracting the second signal from the first signal.

34. The method of claim 32, wherein the potential applied is less than +40 mV vs Ag/AgCl.

35. The method of claim 32, wherein the potential applied is about +5 mV to about −125 mV vs Ag/AgCl.

36. The method of claim 32, wherein the potential applied is about −80 mV vs Ag/AgCl.
